(19) United States
(12) Patent Application Publication
(10) Pub. No.: US 2008/0183282 A1
(43) Pub. Date: Jul. 31, 2008

(54) Use of lipid conjugates for the coating of stents and catheters

(76) Inventor: Saul Yedgar, Jerusalem (IL)

Correspondence Address: Pearl Cohen Zedek Latzer, LLP, 1500 Broadway, 12th Floor, New York, NY, 10036, US

(21) Appl. No.: 11/716,015

(22) Filed: Mar. 9, 2007

Related U.S. Application Data
(60) Provisional application No. 60/780,516, filed on Mar. 9, 2006.

(51) Int. Cl. A61F 2/82

(52) U.S. Cl. 623/1.43; 623/1.46

(57) ABSTRACT

This invention provides inter alia, coated device on at least a portion of a surface of the device. The device coating comprises a lipid or phospholipid moiety bound to a polypyranose. Methods of preventing, inhibiting or treating vessel damage or vessel occlusion, for example in a disease of the vasculature in a subject such as cardiovascular or cerbrovascular disease are described.

### CROSS-REFERENCE TO RELATED APPLICATIONS

This invention claims the benefit of U.S. Provisional Application Ser. No. 60/780,516, filed Mar. 9, 2006, which is hereby incorporated by reference in its entirety.

### FIELD OF THE INVENTION

This invention provides compounds with which implantable devices, including inter-alia, stents and catheters, may be coated, to prevent or treat negative reactions to implantable devices, including inter alia, restenosis and inflammation.

### BACKGROUND OF THE INVENTION

Lipid-conjugates are thought to inhibit the enzyme phospholipase A2 (PLA2, EC 3.1.1.4). Phospholipase A2 catalyzes the breakdown of phospholipids at the sn-2 position to produce a fatty acid and a lysophospholipid. The activity of this enzyme has been correlated with various cell functions, particularly with the production of lipid mediators such as eicosanoid production (prostaglandins, thromboxanes and leukotrienes), platelet activating factor and lysophospholipids. Lipid-conjugates may offer a wider scope of protection of cells and organisms from injurious agents and pathogenic processes.

Invasive medical procedures, such as catheterization of arteries or veins or open surgery, which may be performed for diagnostic and/or therapeutic purposes, are frequently associated with tissue ischemia due to blood vessel injury as well as to reperfusion injury.

Formation of these lesions involves a multiplicity of participants, including coagulative elements of the blood, blood cells, and the structural elements and cells of the blood vessel lumen wall. For example, arterial restenosis appearing after successful balloon angioplasty is frequently due to the narrowing of the inner diameter of the artery by the growth (proliferation) of smooth muscle cells in the areas of irritation caused by the balloon angioplasty. This new stenotic lesion may be comprised from other cell types as well, including leukocytes, accumulating at the lesion site through processes of migration and local proliferation. The two events (cell migration and proliferation) are almost certainly due to the coordinated interaction of a number of different cytokines likely released by early accumulation of macrophages at the site of original tissue injury. Thus, leukocytes contribute to stenotic lesion formation through the processes of migration, local proliferation, passage through endothelial barriers, accumulation of cholesterol-rich lipoprotein, conversion to foam cells, and secretion of cytokines. This proliferation of cells and narrowing of the vascular lumen is not restricted or limited to the coronary arteries or cerebral circulation. It can also occur post-operatively causing restenosis in, for example, peripheral vascular systems.

Implantation of medical devices such as stents, catheters, and cannulas have become commonplace in current medical practice as a way of relieving obstructed blood vessels to allow the passage of blood, oxygen and nutrients. For example, a stent is an expandable wire mesh or hollow perforated tube that is inserted into a hollow structure of the body to keep it open whose main purpose is to overcome decreases in vessel or duct diameter. Stents are often used to reverse or minimize blockade or occlusion of coronary arteries, as well as peripheral arteries and veins, bile ducts, esophagus, trachea or large bronchi, ureters, and urethra.

Prior to deployment, a stent is collapsed into a small diameter; current stents are self-expandable or can be dilated using an inflatable balloon. After expansion, stents are affixed to the vessel or duct wall by their own radial tension. These devices are most commonly inserted under fluoroscopic guidance or endoscopy,

Coronary and peripheral angioplasty is routinely performed to treat obstructive atherosclerotic lesions in the coronary and peripheral blood vessels. Following balloon dilation of these blood vessels, 30-40% of patients undergo restenosis

Catheters are used in a variety of medical applications related to cardiovascular, gastrointestinal, ophthalmic, urolological and urogenital procedures. Catheters are also used for drainage of fluid collections and administration of fluids. Stents are used to diminish pressure differences in flow to or from organs beyond an obstruction in order to maintain adequate flow. Stents are used in blood vessel, bile ducts, respiratory, urolological and urogenital procedures.

Phlebitis, extravasation, allergic-type reactions, obstructive granulation tissue, stenosis at the ends of the stent, stent migration or fracture and infection are among the most frequent complications associated with procedures utilizing mechanical means to ameliorate blockade or occlusion, and to date pose a formidable obstacle to successful implementation in many cases.

### SUMMARY OF THE INVENTION

This invention relates, in one embodiment, to a device having a coating on at least a portion of a surface of the device, wherein the coating comprises a lipid or phospholipid moiety bound to a polypyranose.

In one embodiment, the coating comprises a compound represented by the structure of the general formula (A):

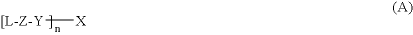

wherein
L is a lipid or a phospholipid;
Z is either nothing, ethanolamine, serine, inositol, choline, phosphate, or glycerol;
Y is either nothing or a spacer group ranging in length from 2 to 30 atoms;
X is a glycosaminoglycan; and
n is a number from 1 to 1000;
wherein any bond between L, Z, Y and X is either an amide or an esteric bond.

In another embodiment, this invention provides, a method of inhibiting or treating vessel damage or vessel occlusion in a subject comprising the step of applying to the vessel a device having a coating on at least a portion of a surface of the device, wherein coating comprising a lipid or phospholipid moiety bound to a polypyranose.

### BRIEF DESCRIPTION OF FIGURES

FIG. 1A: Effect of Compound XXII on unstimulated bovine aortic smooth muscle cell (SMC) proliferation.

FIG. 1B: Effect of Compound XXII on thrombin-stimulated proliferation of bovine aortic SMCs.

FIG. 1C: Effect of Lipid-conjugates on proliferation of human venous smooth muscle cells.

FIG. 1D: Effect of Lipid-conjugates on ischemia/reperfusion—induced leukocyte adhesion (A) and extravasation (B) in rat cremaster muscle.

FIG. 1E: Effect of Lipid-conjugates on red blood cell (RBC) adhesion to activated endothelial cells (EC).

FIG. 2A: Effect of Lipid-conjugates on endogenous low density lipoprotein (LDL)-phospholipase A₂ activity.

FIG. 2B: Effect of Compound XXII on uptake of oxidized low density lipoprotein (oLDL).

FIG. 3A: A Lipid-conjugate protects BGM cells from membrane lysis induced by combined action of hydrogen peroxide produced by glucose oxidase (GO) and exogenous phospholipase A₂ (PLA₂).

FIG. 3B: A Lipid-conjugate protects BGM cells from glycosaminoglycan degradation by hydrogen peroxide produced by glucose oxidase (GO).

FIG. 3C: A Lipid-conjugate protects low density lipoprotein (LDL) from copper-induced oxidation.

FIG. 4: Effect of Compound XXIIf-110 on smooth muscle cells (SMC).

FIG. 5: Effect of Compound XXIII-120 on U937 cells.

FIG. 6: Effect of Compound XXIV-130 on adherence of U937 cells to smooth muscle cells.

FIG. 7: FIG. 7A: Effect of Compound XXV-75 on proliferation of smooth muscle cells (SMC). FIG. 7B: Effect of Compound XXV-75 on proliferation of smooth muscle cells (SMC) cultured with Interleukin-1 (IL-1), platelet derived growth and factor (PDGF).

FIG. 8: Toxicity of Compound XXVIII-90 to smooth muscle cells.

### DETAILED DESCRIPTION OF THE INVENTION

This invention is directed, in some embodiments, to coated devices and methods of use thereof in treating an array of medical conditions, or in other embodiments, for an array of medical applications.

In one embodiment, the invention provides a device having a coating comprising a lipid or phospholipid moiety bound to a physiologically acceptable monomer, dimer, oligomer, or polymer, and/or a pharmaceutically acceptable salt or a pharmaceutical product thereof. In one embodiment, the lipid or phospholipid moiety bound to a physiologically acceptable monomer, dimer, oligomer, or polymer, and/or a pharmaceutically acceptable salt or a pharmaceutical product thereof is referred to as a compound for use in a method and/or device of this invention.

### Compounds

In one embodiment, the compounds for use in any method and/or devices of this invention comprise a lipid or phospholipid moiety bound to a physiologically acceptable monomer, dimer, oligomer, or polymer. In one embodiment, the compounds are also referred to as Lipid-conjugates, and, in some embodiments are described by the general formula:

[phosphatidylethanolamine-Y]n—X

[phosphatidylserine-Y]n—X

[phosphatidylcholine-Y]n—X

[phosphatidylinositol-Y]n—X

[phosphatidylglycerol-Y]n—X

[phosphatidic acid-Y]n—X

[lyso-phospholipid-Y]n—X

[diacyl-glycerol-Y]n—X

[monoacyl-glycerol-Y]n—X

[sphingomyelin-Y]n—X

[sphingosine-Y]n—X

[ceramide-Y]n—X

wherein
Y is either nothing or a spacer group ranging in length from 2 to 30 atoms; and
X is a physiologically acceptable monomer, dimer, oligomer or polymer; and
n is the number of lipid molecules bound to a molecule of X, wherein n is a number from 1 to 1000.

In one embodiment, the invention provides low-molecular weight Lipid-conjugates, previously undisclosed and unknown to possess pharmacological activity, of the general formula described hereinabove. In another embodiment, wherein the general formula described hereinabove describes low-molecular weight Lipid-conjugates, X is a mono- or disaccharide, carboxylated disaccharide, mono- or dicarboxylic acids, a salicylate, salicylic acid, aspirin, lactobionic acid, maltose, an amino acid, glycine, acetic acid, butyric acid, dicarboxylic acid, glutaric acid, succinic acid, fatty acid, dodecanoic acid, didodecanoic acid, bile acid, cholic acid, cholesterylhemmisuccinate, a di- or tripeptide, an oligopeptide, a trisacharide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, or hyaluronic acid.

In one embodiment of this invention, X is salicylate, salicylic acid, aspirin, a monosaccharide, lactobionic acid, maltose, an amino acid, glycine, carboxylic acid, acetic acid, butyric acid, dicarboxylic acid, glutaric acid, succinic acid, fatty acid, dodecanoic acid, didodecanoic acid, bile acid, cholic acid, cholesterylhemmisuccinate, a dipeptide, a disaccharide, a trisaccharide, an oligosaccharide, an oligopeptide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, or hyaluronic acid, a glycosaminoglycan, polygeline (‘haemaccel’), alginate, hydroxyethyl starch (hetastarch), polyethylene glycol, polycarboxylated polyethylene glycol, chondroitin-6-sulfate, chondroitin-4-sulfate, keratin, keratin sulfate, heparan sulfate, dermatin, dermatan sulfate, carboxymethylcellulose, heparin, dextran, or hyaluronic acid.

As defined by the structural formulae provided herein for the Lipid-conjugates, these compounds may contain between one to one thousand lipid moieties bound to a single physiologically acceptable polymer molecule. In one embodiment of this invention, n is a number from 1 to 1000. In another embodiment, n is a number from 1 to 500. In another embodiment, n is a number from 1 to 100. In another embodiment, n is a number from 2 to 1000. In another embodiment, n is a number from 2 to 100. In another embodiment, n is a number from 2 to 200. In another embodiment, n is a number from 3 to 300. In another embodiment, n is a number from 10 to 400. In another embodiment, n is a number from 50 to 500. In another embodiment, n is a number from 100 to 300. In another embodiment, n is a number from 300 to 500. In another embodiment, n is a number from 500 to 800. In another embodiment, n is a number from 500 to 1000.

In one embodiment of the invention, when the conjugated carrier moiety is a polymer, the ratio of lipid moieties covalently bound may range from one to one thousand lipid residues per polymer molecule, depending upon the nature of the polymer and the reaction conditions employed. For example, the relative quantities of the starting materials, or the extent of the reaction time, may be modified in order to obtain Lipid-conjugate products with either high or low ratios of lipid residues per polymer, as desired.

In one embodiment, the set of compounds comprising phosphatidylethanolamine covalently bound to a physiologically acceptable monomer, dimmer, oligomer, or polymer, is referred to herein as the PE-conjugates. In one embodiment, the phosphatidylethanolamine moiety is dipalmitoyl phosphatidylethanolamine. In another embodiment, the phosphatidylethanolamine moiety is dimyristoyl phosphatidylethanolamine. In another embodiment, related derivatives, in which either phosphatidylserine, phosphatidylcholine, phosphatidylinositol, phosphatidic acid or phosphatidylglycerol are employed in lieu of phosphatidylethanolamine as the lipid moiety provide equivalent therapeutic results, based upon the biological experiments described below for the Lipid-conjugates and the structural similarities shared by these compounds.

In another embodiment, the lipid or phospholipid moiety is phosphatidic acid, an acyl glycerol, monoacylglycerol, diacylglycerol, triacylglycerol, sphingosine, sphingomyelin, chondroitin-4-sulfate, chondroitin-6-sulfate, ceramide, phosphatidylethanolamine, phosphatidylserine, phosphatidylcholine, phosphatidylinositol, or phosphatidylglycerol, or an ether or alkyl phospholipid derivative thereof.

Other Lipid-conjugate derivatives relevant to this invention are Lipid-conjugates wherein at least one of the fatty acid groups of the lipid moieties at position C1 or C2 of the glycerol backbone are substituted by a long chain alkyl group attached by amide, ether or alkyl bonds, rather than ester linkages.

In the methods, according to embodiments of the invention, the Lipid-conjugates administered to the subject are comprised from at least one lipid moiety covalently bound through an atom of the polar head group to a monomeric or polymeric moiety (referred to herein as the conjugated moiety) of either low or high molecular weight. When desired, an optional bridging moiety can be used to link the Lipid-conjugates moiety to the monomer or polymeric moiety. The conjugated moiety may be a low molecular weight carboxylic acid, dicarboxylic acid, fatty acid, dicarboxylic fatty acid, acetyl salicylic acid, cholic acid, cholesterylhemisuccinate, or mono- or di-saccharide, an amino acid or dipeptide, an oligopeptide, a glycoprotein mixture, a di- or trisaccharide monomer unit of a glycosaminoglycan such as a repeating unit of heparin, heparan sulfate, hyaluronic acid, chondroitin-sulfate, dermatan, keratan sulfate, or a higher molecular weight peptide or oligopeptide, a polysaccharide, polyglycan, protein, glycosaminoglycan, or a glycoprotein mixture. The composition of phospholipid-conjugates of high molecular weight, and associated analogues, are the subject of U.S. Pat. No. 5,064,817.

In one embodiment, the term “moiety” means a chemical entity otherwise corresponding to a chemical compound, which has a valence satisfied by a covalent bond.

In one embodiment, examples of polymers which can be employed as the conjugated moiety for producing Lipid-conjugates for use in the methods of this invention may be physiologically acceptable polymers, including water-dispersible or -soluble polymers of various molecular weights and diverse chemical types, mainly natural and synthetic polymers, such as glycosaminoglycans, hyaluronic acids, heparin, heparin sulfates, chondroitin sulfates, chondroitin-6-sulfates, chondroitin-4-sulfates, keratins, keratin sulfates, dermatins, dermatan sulfates, dextrans, plasma expanders, including polygeline (“Haemaccel”, degraded gelatin polypeptide cross-linked via urea bridges, produced by “Behring”), “hydroxyethylstarch” (Hetastarch, HES) and extrans, food and drug additives, soluble cellulose derivatives (e.g., methylcellulose, carboxymethylcellulose), polyaminoacids, hydrocarbon polymers (e.g., polyethylene), polystyrenes, polyesters, polyamides, polyethylene oxides (e.g. polyethyleneglycols, polycarboxyethyleneglycols, polycarboxylated polyethyleneglycols), polyvinnylpyrrolidones, polysaccharides, alginates, assimilable gums (e.g., xanthan gum), peptides, injectable blood proteins (e.g., serum albumin), cyclodextrin, and derivatives thereof.

In one embodiment, examples of monomers, dimers, and oligomers which can be employed as the conjugated moiety for producing Lipid-conjugates for use in the methods of the invention may be mono- or disaccharides, trisaccharides, oligopeptides, carboxylic acids, dicarboxylic acids, fatty acids, dicarboxylic fatty acids, salicylates, slicyclic acids, acetyl salicylic acids, aspirins, lactobionic acids, maltoses, amino acids, glycines, glutaric acids, succinic acids, dodecanoic acids, didodecanoic acids, bile acids, cholic acids, cholesterylhemisuccinates, and di- and trisaccharide unit monomers of glycosaminoglycans including heparins, heparan sulfates, hyaluronic acids, chondroitins, chondroitin sulfates, chondroitin-6-sulfates, chondroitin-4-sulfates, dermatins, dermatan sulfates, keratins, keratan sulfates, or dextrans.

In some cases, according to embodiments of the invention, the monomer or polymer chosen for preparation of the Lipid-conjugate may in itself have select biological properties. For example, both heparin and hyaluronic acid are materials with known physiological functions. In the present invention, however, the Lipid-conjugates formed from these substances as starting materials display a new and wider set of pharmaceutical activities than would be predicted from administration of either heparin or hyaluronic acid which have not been bound by covalent linkage to a phospholipid. It can be shown, by standard comparative experiments as described below and in U.S. application Ser. No. 10/952,496, incorporated herein by reference, that phosphatidylethanolamine (PE) linked to hyaluronic acid (Compound XXII), to heparin (Compound XXIV), to chondroitin sulfate A (Compound XXV), to carboxymethylcellulose (Compound XXVI), to Polygeline (haemaccel) (Compound XXVII), or to hydroxyethylstarch (Compound XXVIII), are far superior in terms of potency and range of useful pharmaceutical activity to the free conjugates (the polymers above and the like). In fact, these latter substances are, in general, not considered useful in methods for treatment of most of the diseases described herein, including the treatment of pathogenic infections. Thus, the combination of a phospholipid such as phosphatidylethanolamine, or related phospholipids which differ with regard to the polar head group, such as phosphatidylserine (PS), phosphatidylcholine (PC), phosphatidylinositol (PI), and phosphatidylglycerol (PG), results in the formation of a compound which has novel pharmacological properties when compared to the starting materials alone.

The biologically active Lipid-conjugates described herein can have a wide range of molecular weight, e.g., above 50,000 (up to a few hundred thousands) when it is desirable to retain the Lipid conjugate in the vascular system and below 50,000 when targeting to extravascular systems is desirable. The sole limitation on the molecular weight and the chemical structure of the conjugated moiety is that it does not result in a Lipid-conjugate devoid of the desired biological activity, or lead to chemical or physiological instability to the extent that the Lipid-conjugate is rendered useless as a drug in the method of use described herein.

In one embodiment, the compound for use in the methods and/or devices of the present invention is represented by the structure of the general formula (A):

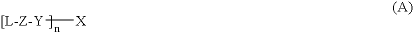

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer; and
wherein X is a glycosaminoglycan; and
n is a number from 1 to 1000;
wherein any bond between L, Z, Y and X is either an amide or an esteric bond.

in one embodiment, L is phosphatidyl, Z is ethanolamine, wherein L and Z are chemically bonded resulting in phosphatidylethanolamine, Y is nothing, and X is carboxymethylcellulose. In one embodiment, the phosphatidylethanolamine moiety is dipalmitoyl phosphatidylethanolamine. In another embodiment, the phosphatidylethanolamine moiety is dimyristoyl phosphatidylethanolamine. In another embodiment X is hydroxyethylstarch (HES). In another embodiment, X is alginate, In another embodiment X is dextran. In another embodiment X is polygeline (‘haemaccel).

In another embodiment, the compound for use in the present invention is represented by the structure of the general formula (I):

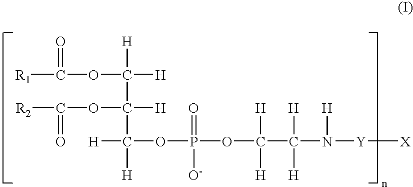

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms; and
X is either a physiologically acceptable monomer, dimer, oligomer or a physiologically acceptable polymer wherein X is a glycosaminoglycan; and
n is a number from 1 to 1,000;
wherein if Y is nothing the phosphatidylethanolamine is directly linked to X via an amide bond and if Y is a spacer, the spacer is directly linked to X via an amide or an esteric bond and to the phosphatidylethanolamine via an amide bond.

In one embodiment, compounds for use in the methods of the invention comprise one of the following as the conjugated moiety X: acetate, butyrate, glutarate, succinate, dodecanoate, didodecanoate, maltose, lactobionic acid, dextran, alginate, hydroxyethylstarch (HES), aspirin, cholate, cholesterylhemisuccinate, carboxymethyl-cellulose, heparin, hyaluronic acid, chondroitin sulfate, polygeline (haemaccel), polyethyleneglycol, and polycarboxylated polyethylene glycol. The polymers used as starting material to prepare the PE-conjugates may vary in molecular weight from 1 to 2,000 kDa.

Examples of phosphatidylethanolamine (PE) moieties are analogues of the phospholipid in which the chain length of the two fatty acid groups attached to the glycerol backbone of the phospholipid varies from 2-30 carbon atoms length, and in which these fatty acids chains contain saturated and/or unsaturated carbon atoms. In lieu of fatty acid chains, alkyl chains attached directly or via an ether linkage to the glycerol backbone of the phospholipid are included as analogues of PE. In one embodiment, the PE moiety is dipalmitoyl-phosphatidyl-ethanolamine. In another embodiment, the PE moiety is dimyristoyl-phosphatidyl-ethanolamine.

Phosphatidyl-ethanolamine and its analogues may be from various sources, including natural, synthetic, and semisynthetic derivatives and their isomers.

Phospholipids which can be employed in lieu of the PE moiety are N-methyl-PE derivatives and their analogues, linked through the amino group of the N-methyl-PE by a covalent bond; N,N-dimethyl-PE derivatives and their analogues linked through the amino group of the N,N-dimethyl-PE by a covalent bond, phosphatidylserine (PS) and its analogues, such as palmitoyl-stearoyl-PS, natural PS from various sources, semisynthetic PSs, synthetic, natural and artifactual PSs and their isomers. Other phospholipids useful as conjugated moieties in this invention are phosphatidylcholine (PC), phosphatidylinositol (PI), phosphatidic acid and phosphoatidylglycerol (PG), as well as derivatives thereof comprising either phospholipids, lysophospholipids, phosphatidyic acid, sphingomyelins, lysosphingomyelins, ceramide, and sphingosine.

For PE-conjugates and PS-conjugates, the phospholipid is linked to the conjugated monomer or polymer moiety through the nitrogen atom of the phospholipid polar head group, either directly or via a spacer group. For PC, PI, and PG conjugates, the phospholipid is linked to the conjugated monomer or polymer moiety through either the nitrogen or one of the oxygen atoms of the polar head group, either directly or via a spacer group.

In another embodiment, a compound for use in the methods and/or devices of the present invention is represented by the structure of the general formula (II):

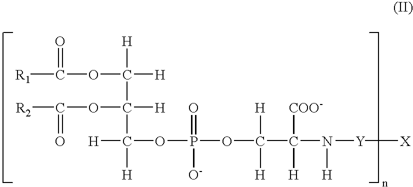

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is a physiologically acceptable monomer, dimer, oligomer or polymer wherein
X is a glycosaminoglycan; and
n is a number from 1 to 1000;
wherein if Y is nothing, the phosphatidylserine is directly linked to X via an amide bond and if Y is a spacer, the spacer is directly linked to X via an amide or an esteric bond and to the phosphatidylserine via an amide bond.

In one embodiment, the phosphatidylserine may be bonded to Y, or to X if Y is nothing, via the COO⁻ moiety of the phosphatidylserine.

In another embodiment, a compound for use in the present invention is represented by the structure of the general formula (III):

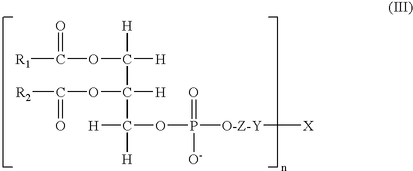

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phosphatidyl, Z, Y and X is either an amide or an esteric bond.

In another embodiment, the compound for use in the present invention is represented by the structure of the general formula (IV):

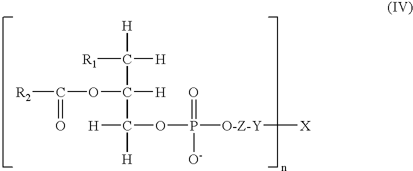

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer
wherein X is a glycosaminoglycan; and
n is a number from 1 to 1000;
wherein any bond between the phospholipid, Z, Y and X is either an amide or an esteric bond.

In another embodiment, the compound for use in the present invention is represented by the structure of the general formula (V):

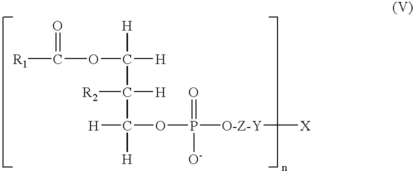

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In another embodiment, the compound for use in the present invention is represented by the structure of the general formula (VI):

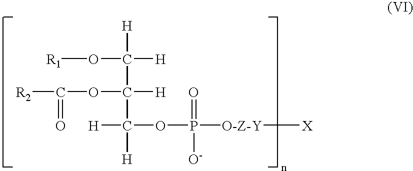

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In another embodiment, the compound for use in the present invention is represented by the structure of the general formula (VII):

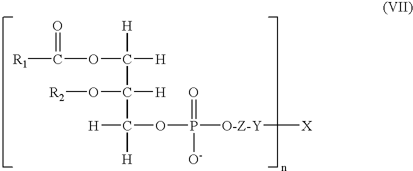

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In one embodiment of the invention, phosphatidylcholine (PC), phosphatidylinositol (PI), phosphatidic acid (PA), wherein Z is nothing, and phosphatidylglycerol (PG) conjugates are herein defined as compounds of the general formula (III).

In another embodiment, the compound for use in the present invention is represented by the structure of the general formula (VIII):

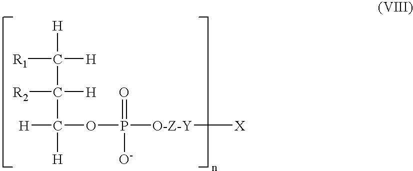

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In another embodiment, the compound for use in the present invention is represented by the structure of the general formula (IX):

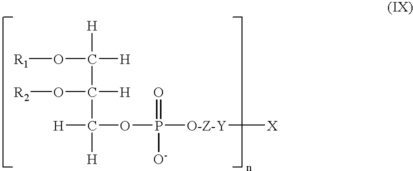

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In another embodiment, the compound for use in the present invention is represented by the structure of the general formula (IXa):

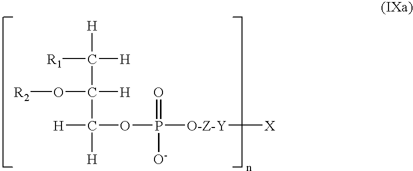

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In another embodiment, the compound for use in the present invention is represented by the structure of the general formula (IXb):

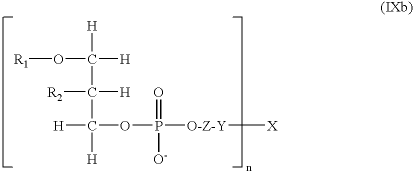

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In another embodiment, the compound for use in the present invention is represented by the structure of the general formula (X):

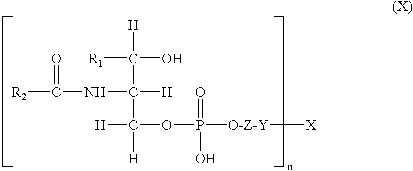

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the ceramide phosphoryl, Z, Y and X is either an amide or an esteric bond.

In another embodiment, the compound for use in the present invention is represented by the structure of the general formula (XI):

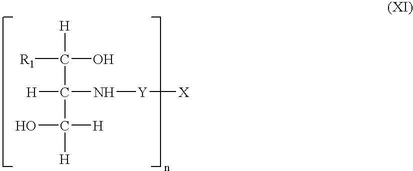

wherein
R₁ is a linear, saturated, mono-unsaturated, or poly-unsaturated, alkyl chain ranging in length from 2 to 30 carbon atoms;
Y is either nothing or a spacer group ranging in length from 2 to 30 atoms;
X is a physiologically acceptable monomer, dimer, oligomer or polymer wherein
X is a glycosaminoglycan; and
n is a number from 1 to 1000;
wherein if Y is nothing the sphingosyl is directly linked to X via an amide bond and if Y is a spacer, the spacer is directly linked to X and to the sphingosyl via an amide bond and to X via an amide or an esteric bond.

In another embodiment, the compound for use in the present invention is represented by the structure of the general formula (XII):

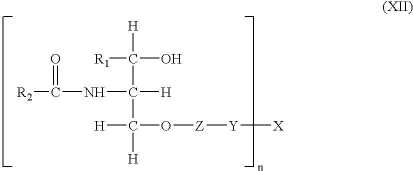

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer or polymer wherein
X is a glycosaminoglycan; and
n is a number from 1 to 1000;
wherein any bond between the ceramide, Z, Y and X is either an amide or an esteric bond.

In another embodiment, the compound for use in the present invention is represented by the structure of the general formula (XIII):

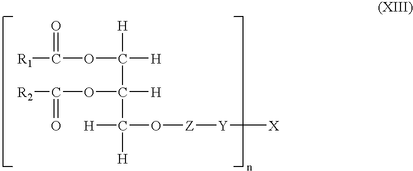

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein
X is a glycosaminoglycan; and
n is a number from 1 to 1000;
wherein any bond between the diglyceryl, Z, Y and X is either an amide or an esteric bond.

In another embodiment, the compound for use in the present invention is represented by the structure of the general formula (XIV):

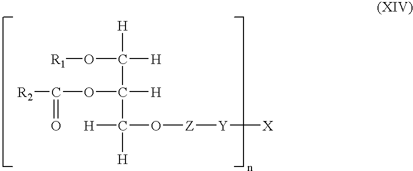

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein
X is a glycosaminoglycan; and
n is a number from 1 to 1000;
wherein any bond between the glycerolipid, Z, Y and X is either an amide or an esteric bond.

In another embodiment, the compound for use in the present invention is represented by the structure of the general formula (XV):

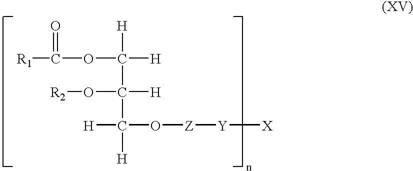

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein
X is a glycosaminoglycan; and
n is a number from 1 to 1000;
wherein any bond between the glycerolipid, Z, Y and X is either an amide or an esteric bond.

In another embodiment, the compound for use in the present invention is represented by the structure of the general formula (XVI):

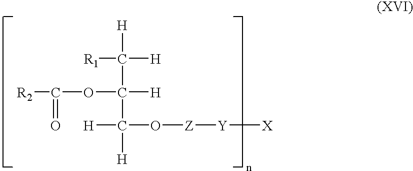

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
V is either nothing or a spacer group ranging in length from 2 to 30 atoms;
X is a physiologically acceptable monomer, dimer, oligomer or polymer wherein
X is a glycosaminoglycan; and
n is a number from 1 to 1000;
wherein any bond between the lipid, Z, Y and X is either an amide or an esteric bond.

In another embodiment, the compound for use in the present invention is represented by the structure of the general formula (XVII):

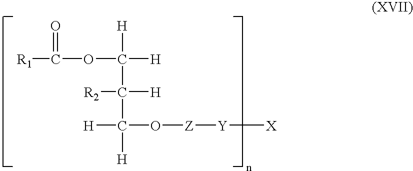

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein
X is a glycosaminoglycan; and
n is a number from 1 to 1000;
wherein any bond between the lipid, Z, Y and X is either an amide or an esteric bond.

In another embodiment, the compound for use in the present invention is represented by the structure of the general formula (XVIII):

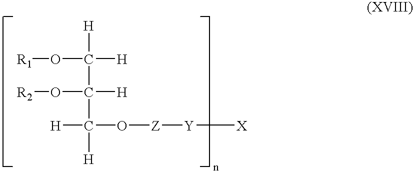

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein any bond between the lipid, Z, Y and X is either an amide or an esteric bond.

In another embodiment, the compound for use in the present invention is represented by the structure of the general formula (XIX):

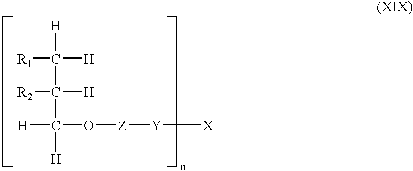

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein any bond between the lipid, Z, Y and X is either an amide or an esteric bond.

In another embodiment, the compound for use in the present invention is represented by the structure of the general formula (XX):

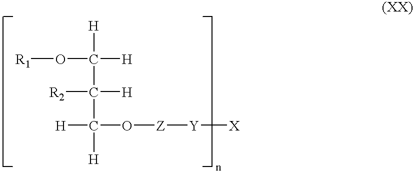

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;

wherein any bond between the lipid, Z, Y and X is either an amide or an esteric bond.

In another embodiment, the compound for use in the present invention is represented by the structure of the general formula (XXI):

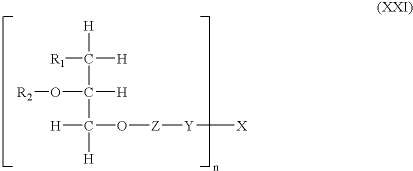

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein any bond between the lipid, Z, Y and X is either an amide or an esteric bond.

For any or all of the compounds represented by the structures of the general formulae (A), (I), (II), (III), (IV), (V), (VI), (VII), (VIII), (IX), (IXa), (IXb), (X), (XI), (XII), (XIII), (XIV), (XV), (XVI), (XVII), (XVIII), (XIX), (XX), (XXI), and (XXII) hereinabove: In one embodiment, X is a glycosaminoglycan. In one embodiment of the invention, the glycosaminoglycan may be, inter alia, hyaluronic acid, heparin, heparan sulfate, chondroitin sulfate, keratin, keratan sulfate, dermatan sulfate or a derivative thereof.

In another embodiment, the glycosaminoglycan is a polymer of disaccharide units. In another embodiment, the number of the disaccharide units in the polymer is m. In another embodiment, m is a number from 2-10,000. In another embodiment, m is a number from 2-500. In another embodiment, m is a number from 2-1000. In another embodiment, m is a number from 50-500. In another embodiment, m is a number from 2-2000. In another embodiment, m is a number from 500-2000. In another embodiment, m is a number from 1000-2000. In another embodiment, m is a number from 2000-5000. In another embodiment, m is a number from 3000-7000. In another embodiment, m is a number from 5000-10,000. In another embodiment, a disaccharide unit of a glycosaminoglycan may be bound to one lipid or phospholipid moiety. In another embodiment, each disaccharide unit of the glycosaminoglycan may be bound to zero or one lipid or phospholipid moieties. In another embodiment, the lipid or phospholipid moieties are bound to the —COOH group of the disaccharide unit. In another embodiment, the bond between the lipid or phospholipid moiety and the disaccharide unit is an amide bond.

In another embodiment, the chondroitin sulfate may be, inter alia, chondroitin-6-sulfate, chondroitin-4-sulfate or a derivative thereof.

In one embodiment of the invention, Y is nothing. Non limiting examples of suitable divalent groups forming the optional bridging group (spacer) Y, according to embodiments of the invention, are straight or branched chain alkylene, e.g., of 2 or more, preferably 4 to 30 carbon atoms, —CO-alkylene-CO, —NH-alkylene-NH—, —CO-alkylene-NH—, —NH-alkylene-NH, CO-alkylene-NH—, an amino acid, cycloalkylene, wherein alkylene in each instance, is straight or branched chain and contains 2 or more, preferably 2 to 30 atoms in the chain, —(—O—CH(CH₃)CH₂—)ₓ— wherein x is an integer of 1 or more.

According to embodiments of the invention, in addition to the traditional phospholipid structure, related derivatives for use in this invention are phospholipids modified at the C1 or C2 position to contain an amine, ether or alkyl bond instead of an ester bond. In one embodiment of the invention, the alkyl phospholipid derivatives and ether phospholipid derivatives are exemplified herein.

In one embodiment of the invention, the sugar rings of the glycosaminoglycan are intact. In another embodiment, intact refers to closed. In another embodiment, intact refers to natural. In another embodiment, intact refers to unbroken.

In one embodiment of the invention, the structure of the lipid or phospholipid in any compound according to the invention is intact. In another embodiment, the natural structure of the lipid or phospholipids in any compound according to the invention is maintained.

In one embodiment, the compounds for use in the present invention are biodegradable.

In one embodiment, the compound according to the invention is phosphatidylethanolamine bound to aspirin. In one embodiment, the compound according to the invention is phosphatidylethanolamine bound to glutarate.

In some embodiments, the compounds for use are as listed in Table 1 below.

TABLE 1

| Phospho- | | | |
|---|---|---|---|
| lipid | Spacer | Polymer (m.w.) | Compound |
| PE | None | Hyaluronic acid | XXII |
| | | (2-2000 kDa) | |
| Dimyristoyl- | None | Hyaluronic acid | XXIII |
| PE | | | |
| PE | None | Heparin | XXIV |
| | | (0.5-110 kDa) | |
| PE | None | Chondroitin sulfate A | XXV |
| PE | None | Carboxymethylcellulose | XXVI |
| | | (20-500 kDa) | |
| PE | Dicarboxylic | Polygeline (haemaccel) | XXVII |
| | acid + | (4-40 kDa) | |
| | Diamine | | |
| PE | None | Hydroxyethylstarch | XXVIII |
| PE | Dicarboxylic | Dextran | XXIX |
| | acid + | (1-2,000 kDa) | |
| | Diamine | | |
| PE | None | Aspirin | XXX |
| PE | Carboxyl amino | Hyaluronic acid | XXXI |
| | group | (2-2000 kDa) | |
| PE | Dicarboxyl group | Hyaluronic acid | XXXII |
| | | (2-2000 kDa) | |
| PE | Dipalmitoic acid | Hyaluronic acid | XXXIII |
| | | (2-2000 kDa) | |
| PE | Carboxyl amino | Heparin | XXXIV |
| | group | (0.5-110 kDa) | |
| PE | Dicarboxyl group | Heparin | XXXV |
| | | (0.5-110 kDa) | |
| PE | Carboxyl amino | Chondroitin sulfate A | XXXVI |
| | group | | |
| PE | Dicarboxyl group | Chondroitin sulfate A | XXXVII |
| PE | Carboxyl amino | Carboxymethylcellulose | XXXVIII |
| | group | (20-500 kDa) | |
| PE | Dicarboxyl group | Carboxymethylcellulose | XXXIX |
| | | (20-500 kDa) | |
| PE | None | Polygeline (haemaccel) | XL |
| | | (4-40 kDa) | |
| PE | Carboxyl amino | Polygeline (haemaccel) | XLI |
| | group | (4-40 kDa) | |
| PE | Dicarboxyl group | Polygeline (haemaccel) | XLII |
| | | (4-40 kDa) | |
| PE | Carboxyl amino | Hydroxyethylstarch | XLIII |
| | group | | |
| PE | Dicarboxyl group | Hydroxyethylstarch | XLIV |
| PE | None | Dextran | XLV |
| | | (1-2,000 kDa) | |
| PE | Carboxyl amino | Dextran | XLVI |
| | group | (1-2,000 kDa) | |
| PE | Dicarboxyl group | Dextran | XLVII |
| | | (1-2,000 kDa) | |
| PE | Carboxyl amino | Aspirin | XLVIII |
| | group | | |
| PE | Dicarboxyl group | Aspirin | XLIX |
| PE | None | Albumin | L |
| PE | None | Alginate | LI |
| | | (2-2000 kDa) | |
| PE | None | Polyaminoacid | LII |
| PE | None | Polyethylene glycol | LIII |
| PE | None | Lactobionic acid | LIV |
| PE | None | Acetylsalicylate | LV |
| PE | None | Cholesteryl- | LVI |
| | | hemmisuccinate | |
| PE | None | Maltose | LVII |
| PE | None | Cholic acid | LVIII |
| PE | None | Chondroitin sulfates | LIX |
| PE | None | Polycarboxylated | LX |
| | | polyethylene glycol | |
| Dipalmitoyl- | None | Hyaluronic acid | LXI |
| PE | | | |
| Dipalmitoyl- | None | Heparin | LXII |
| PE | | | |
| Dipalmitoyl- | None | Chondroitin sulfate A | LXIII |
| PE | | | |
| Dipalmitoyl- | None | Carboxymethylcellulose | LXIV |
| PE | | | |
| Dipalmitoyl- | None | Polygeline (haemaccel) | LXV |
| PE | | | |
| Dipalmitoyl- | None | Hydroxyethylstarch | LXVI |
| PE | | | |
| Dipalmitoyl- | None | Dextran | LXVII |
| PE | | | |
| Dipalmitoyl- | None | Aspirin | LXVIII |
| PE | | | |
| Dimyristoyl- | None | Heparin | LXVIX |
| PE | | | |
| Dimyristoyl- | None | Chondroitin sulfate A | LXX |
| PE | | | |
| Dimyristoyl- | None | Carboxymethylcellulose | LXXI |
| PE | | | |
| Dimyristoyl- | None | Polygeline (haemaccel) | LXXII |
| PE | | | |
| Dimyristoyl- | None | Hydroxyethylstarch | LXXIII |
| PE | | | |
| Dimyristoyl- | None | Dextran | LXXIV |
| PE | | | |
| Dimyristoyl- | None | Aspirin | LXXV |
| PE | | | |
| PS | None | Hyaluronic acid | LXXVI |
| PS | None | Heparin | LXXVII |
| PS | None | Polygeline (haemaccel) | LXXVIII |
| PC | None | Hyaluronic acid | LXXIX |
| PC | None | Heparin | LXXX |
| PC | None | Polygeline (haemaccel) | LXXXI |
| PI | None | Hyaluronic acid | LXXXII |
| PI | None | Heparin | LXXXIII |
| PI | None | Polygeline (haemaccel) | LXXXIV |
| PG | None | Hyaluronic acid | LXXXV |
| PG | None | Heparin | LXXXVI |
| PG | None | Polygeline (haemaccel) | LXXXVII |
| PE | None | Glutaryl | LXXXVIII |

In another embodiment, the compounds for use in this invention are: XXIIf-110, XXIIt-110, XXIII-120, XXIV-130, XXV-75, XXV-100, LI-120, XL80, XXVIII-90, XLV-40/100, XLV-10/60 or XLV-5/60.

In one embodiment of the invention, the compounds coating devices of this invention and/or uses thereof are Compound XXII, Compound XXIII, Compound XXIV, Compound XXV, Compound XXVI, Compound XXVII, Compound XXVIII, Compound XXIX, Compound XXX, or pharmaceutically acceptable salts thereof, in combination with a physiologically acceptable carrier or solvent. According to embodiments of the invention, these polymers, when chosen as the conjugated moiety, may vary in molecular weights from 200 to 2,000,000 Daltons. In one embodiment of the invention, the molecular weight of the polymer as referred to herein is from 200 to 1000 Daltons. In another embodiment, the molecular weight of the polymer as referred to herein is from 200 to 1000 Daltons. In another embodiment, the molecular weight of the polymer as referred to herein is from 1000 to 5000 Daltons. In another embodiment, the molecular weight of the polymer as referred to herein is from 5000 to 10,000 Daltons. In another embodiment, the molecular weight of the polymer as referred to herein is from 10,000 to 20,000 Daltons. In another embodiment, the molecular weight of the polymer as referred to herein is from 10,000 to 50,000 Daltons. In another embodiment, the molecular weight of the polymer as referred to herein is from 20,000 to 70,000 Daltons. In another embodiment, the molecular weight of the polymer as referred to herein is from 50,000 to 100,000 Daltons. In another embodiment, the molecular weight of the polymer as referred to herein is from 100,000 to 200,000 Daltons. In another embodiment, the molecular weight of the polymer as referred to herein is from 200,000 to 500,000 Daltons. In another embodiment, the molecular weight of the polymer as referred to herein is from 200,000 to 1,000,000 Daltons. In another embodiment, the molecular weight of the polymer as referred to herein is from 500,000 to 1,000,000 Daltons. In another embodiment, the molecular weight of the polymer as referred to herein is from 1,000,000 to 2,000,000 Daltons. Various molecular weight species have been shown to have the desired biological efficacy, as shown in the section below.

In one embodiment of this invention, low molecular weight Lipid-conjugates are defined hereinabove as the compounds of formula (I)-(XXI) wherein X is a mono- or disaccharide, carboxylated disaccharide, mono- or dicarboxylic acids, a salicylate, salicylic acid, aspirin, lactobionic acid, maltose, an amino acid, glycine, acetic acid, butyric acid, dicarboxylic acid, glutaric acid, succinic acid, fatty acid, dodecanoic acid, didodecanoic acid, bile acid, cholic acid, cholesterylhemmisuccinate, a di- or tripeptide, an oligopeptide, a trisacharide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, carboxymethylcellulose, alginate, polygeline (haemaccel), hydroxyethylstarch (HES) or hyaluronic acid.

Examples of suitable divalent groups forming the optional bridging group Y are straight- or branched-chain alkylene, e.g., of 2 or more, preferably 4 to 18 carbon atoms, —CO—alkylene-CO, —NH-alkylene-NH—, —COalkylene-NH—, cycloalkylene, wherein alkylene in each instance, is straight or branched chain and contains 2 or more, preferably 2 to 18 carbon atoms in the chain, —(—O—CH(CH₃)CH₂—)ₓ— wherein x is an integer of 1 or more.

In another embodiment, in addition to the traditional phospholipid structure, related derivatives for use in this invention are phospholipids modified at the C1 or C2 position to contain an ether or alkyl bond instead of an ester bond. These derivatives are exemplified hereinabove by the general formulae (VIII) and (IX).

In one embodiment of the invention, X is covalently conjugated to a lipid. In another embodiment, X is covalently conjugated to a lipid via an amide bond. In another embodiment, X is covalently conjugated to a lipid via an esteric bond. In another embodiment, the lipid is phosphatidylethanolamine.

Cell surface GAGs play a key role in protecting cells from diverse damaging agents and processes, such as reactive oxygen species and free radicals, endotoxins, cytokines, invasion promoting enzymes, and agents that induce and/or facilitate degradation of extracellular matrix and basal membrane, cell invasiveness, white cell extravasation and infiltration, chemotaxis, and others. In addition, cell surface GAGs protect cells from bacterial, viral and parasitic infection, and their stripping exposes the cell to interaction and subsequent internalization of the microorganism. Enrichment of cell surface GAGs would thus assist in protection of the cell from injurious processes. Thus, in one embodiment of the invention, PLA2 inhibitors are conjugated to GAGs or GAG-mimicking molecules. In another embodiment, these Lipid-conjugates provide wide-range protection from diverse injurious processes, and are effective in amelioration of diseases that requires cell protection from injurious biochemical mediators.

In another embodiment, a GAG-mimicking molecule may be, inter alia, a negatively charged molecule. In another embodiment, a GAG-mimicking molecule may be, inter alia, a salicylate derivative. In another embodiment, a GAG-mimicking molecule may be, inter alia, a dicarboxylic acid.

In another embodiment, the invention provides a device coated on at least a portion of a surface of the device, with the compounds described herein, wherein a coating is applied to the portion of the surface of the device, which comprises the compounds as herein described. In one embodiment, such a coating may comprise a composition including, inter-alia, a pharmaceutically acceptable carrier or excipient and the compounds as herein described.

### Preparation of Compounds for Use in the Present Invention

The preparation of some high molecular weight Lipid-conjugates is the subject of U.S. Pat. No. 5,064,817, which is incorporated herein by reference. These synthetic methods are considered to be applicable as well to the preparation of low molecular weight Lipid-conjugates, i.e. Lipid-conjugates comprising monomers and dimers as the conjugated moiety, with appropriate modifications in the procedure as would be readily evident to one skilled in the art. The preparation of some low molecular weight Lipid-conjugates may be conducted using methods well known in the art or as described in U.S. patent application Ser. No. 10/952,496, which is incorporated herein by reference in its entirety. Example 10 describes the preparation of representative lipid conjugates.

Any method may be utilized to ascertain synthesis of the respective compounds, as will be known to one skilled in the art. For example, and in one embodiment, high pressure liquid chromatography (HPLC) in reverse or direct phase, gas chromatography (GC), mass spectrometry, and other methods may be used.

The compounds as described herein, are applied to at least a portion of a surface of a device of this invention.

In one embodiment, the application of a compound as herein described a device of this invention may be referred to as “device coating” or in other embodiments, the compound, when present in any region of a surface of the device may be referred to, in one embodiment as “a coating”. In one embodiment, the term “coating” refers to such application, where the compound remains in association with at least a portion of a surface of a device, for a period of time, which may range from seconds to years, as will be suitable for a given application.

### Coatings

In one embodiment of the present invention, the term “coating” refers to the applied compound on at least a portion of a surface of the device. In one embodiment, the term “coating” refers to an association of at least one compound, as described herein, with at least a portion of a surface, or in another embodiment, an entire surface, or in another embodiment, two or more portions of a surface, or in another embodiment, two or more surfaces, or in another embodiment, two or more portions of two or more surfaces, etc. In some embodiments, coating refers to associations that are transient, or in another embodiment, permanent.

In one embodiment, association is by means of chemical conjugation. In one embodiment, the association is via physical entrapment. In another embodiment, coating is a result of both chemical conjugation and physical entrapment. In some embodiments, such associations may be via covalent bonding, or in another embodiment, ionic bonding, or in another embodiment, hydrophobic interations, or in another embodiment, via Van Der Waal's forces, etc., or any appropriate interaction, as will be appreciated by one skilled in the art.

In one embodiment, association is by means of a cross-linkable polymer. In one embodiment, a homopolymer such as acrylic polymer or epoxy polymer containing one or more functional groups is used. In some embodiments, association is achieved using a copolymer such as polyurethane, polyamide, or polyester containing one or more functional groups. In one embodiment, functional groups comprise carboxylate group, hydroxyl group, amine group, or epoxy group.

In one embodiment a buffer agent can further interact with the polymer via covalent bonding, hydrogen bonding, or ionic bonding, thereby further prolonging the coated device's resistance to pH change. In one embodiment, the buffer agent can also be linked, via covalent bonding, hydrogen bonding or ionic bonding, to a functionalized or ionized surface of the device. In one embodiment, a hydrophilic polymer is included in a coating of a device of this invention.

In one embodiment, the agent promoting the association is a polysaccharide. In one embodiment, it is a mucopolysaccharide or glycosaminoglycan. In other embodiments, Hyaluronan, chondroitin sulfate, keratin sulfate, heparan sulfate or dermatan sulfate are used as the agent.

In one embodiment, the device is coated on at least one exposed surface of the device. In one embodiment of the present invention the coating is applied at a particular position on the device as will be known to one skilled in the art. In one embodiment, 10-99% of the device surface area is coated. In another embodiment, 20-50% of the device area is coated.

In one embodiment, the device is coated in an ordered pattern. In one embodiment, the phrase “ordered pattern” refers to a repetitive arrangement. In one embodiment, coating is in a staggered conformation. In one embodiment coating is applied in a spotted pattern. In one embodiment, the spots may be arranged perpendicular with respect to each other, or in another embodiment, in parallel. In another embodiment, spots might radiate outward from a single point, as spikes on a wheel. In one embodiment, the coating is applied to the interior of the device or in one embodiment, the coating is applied on the exterior of the device, or in another embodiment, a combination thereof, and in another embodiment, with any conceivable pattern of deposition, for example, as described herein. In one embodiment, coating will be applied to regions of the device in maximal contact with a cell of a body, or body fluids of a subject in which the device is implanted. In one embodiment, the coating position and/or orientation will be applied as a function of the desired release time as will be known to one skilled in the art.

In one embodiment, application of the coating is such, that coating may be continually, or periodically applied, in some embodiments, or in another embodiment, coating is a dynamic process. For example, at specified times, or throughout the life of the application of the device, the coating may be applied. In one embodiment, coating will be applied from a reservoir connected to the device. In one embodiment, coating will be applied remotely from the reservoir and onto the device. In one embodiment, coating will be applied remotely from a reservoir in a controlled manner.

For example, and in one embodiment, in the case of the device being a catheter; a drip solution can be attached to the catheter delivering the desired amount of compound to the catheter.

In one embodiment, additional materials are being applied. In one embodiment, these materials will further support coating. In one embodiment, the coating of the device with a compound of this invention may employ the use of adhesive compounds likelihood for occlusion of the vasculature into which the coated device, for example, the coated stent is applied.

In one embodiment, the coating may further comprise a compound having an anti-infective effect, such as the compounds described for use herein. In one embodiment, the coating further comprises other anti-infectives, such as, for example, antibiotics comprising: aminoglycosides cephalosporins, antifungals, fungicides, chloramphenicols, macrolides, erythromycins, penicillins, tetracyclines, antivirals or antimalarial agents etc. may be applied, in addition to the compounds as herein described.

In one embodiment of the present invention, the coating may further comprise a compound having an anticoagulant effect. In some embodiments, the anticoagulant is a fibrinolytic agents or a platelet antagonists In one embodiment, the anticoagulant agent is heparin, which is conjugated to a lipid or phospholipid, as herein described, and thus represents a compound for use in this invention.

In one embodiment, the coating further comprises a compound having a chelating effect. In some embodiments, the chelating agent is DIPA acid, EDTA acid, HEDTA acid or NTA acid.

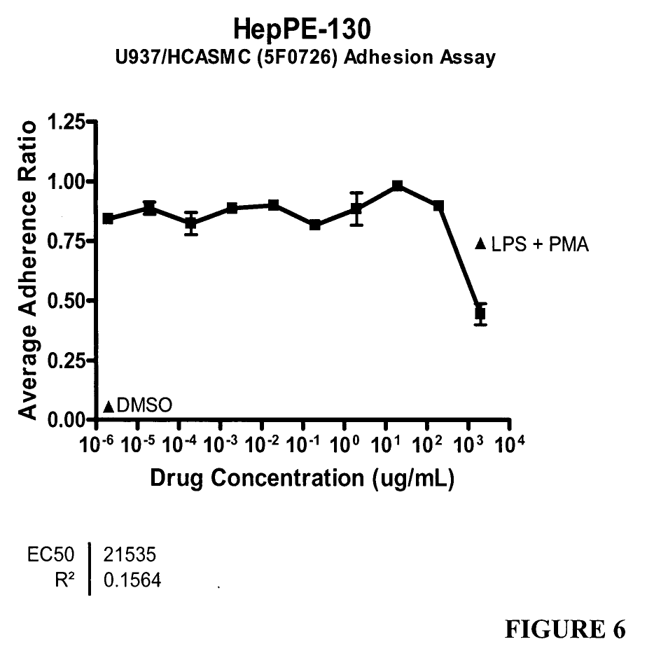

In some embodiments, coating of the devices reduces cell adhesion to such devices. For example, as demonstrated herein, in Example 7 and FIG. 6, the lipid conjugates diminished adherence of U937 cells to smooth muscle cells, thus the coated devices may diminish adhesion of such immune cells or other local cells to the device, or in some embodiments, to distal sites from that of device implantation, such as for example, immune cell adhesion to vasculature at the site of coated stent implantation, for example. In some embodiments, the coated materials further suppress inflammation at the site of implantation, and/or in some embodiments, cell proliferation at such sites. In some embodiments, the coated devices further incorporate, or are co-administered with compounds which synergize to enhance such desired effects, such as suppression of inflammation, exertion of anti-proliferative effects, or suppression of localized cell adhesion.

In one embodiment, an agent, which exerts an antiproliferative effect is incorporated in the device, or in another embodiment, is administered prior to or concurrent with implantation of a coated device of this invention. In some embodiments, the antiproliferative agent is a taxane. In one embodiment, the taxane is paclitaxel. In one embodiment, the antiproliferative agents is doxorubicin. In other embodiments, antiproliferative agents such as compounds that interfere with cyclin-dependent kinase/cyclin holoenzymes, growth factors and transcription factors that control cell cycle progression, which can comprise the coating.

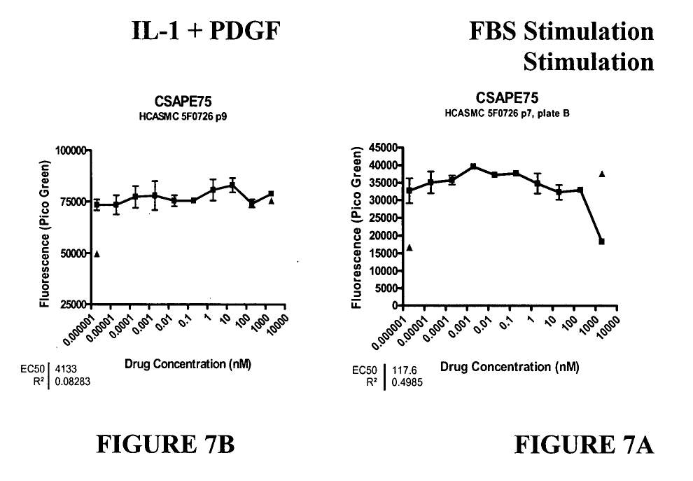

In one embodiment, antiproliferative effects are particularly exerted on vasculture muscle cell or endothelial cell proliferation, for example as demonstrated herein in Example 8 and FIG. 7.

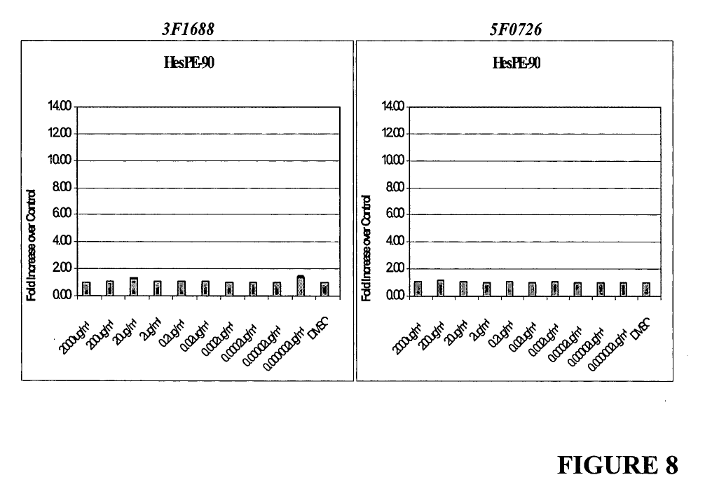

In one embodiment, the coating comprises an agent which exerts no toxic effect on any cells of a subject in contact with coated devices of this invention. In some embodiments, coating is accomplished utilizing concentrations of the coating agent, which exhibit little to no toxicity, for example as shown herein in Example 9 and FIG. 8, where concentrations of even up to 40,000 nM demonstrated little to no toxic effects.

In one embodiment of the present invention, the coating comprises the compounds as herein described, wherein the compound is incorporated within a matrix, which is applied to a portion of a surface of the device. Such adsorption, in one embodiment, affects the release rate of the compound, so as to promote, in some embodiments, immediate release, or in other embodiments release over an extended period. In another embodiment, the coating comprises compounds so adsorbed as to be surface exposed on the applied region of the device.

In one embodiment, the coating comprises the compound adsorbed to a polymer, biopolymer or a silica gel. In one embodiment the term “biopolymer” refers to polymers based on renewable raw materials, which in some embodiments are readily biodegradable or, in other embodiments, not readily biodegradable, e.g. cellulose, or synthetic polymers which are biodegradable, e.g. polylactides. In another embodiment, the polymer is nonbiodegradable.

In one embodiment, the device can be further coated with a polymer. In one embodiment the choice of polymer affects the kinetics of release of the compound. In one embodiment, the choice of polymer is to affect the surface characteristics of the device to suit a desired application. In one embodiment, the polymer is polyethylene terephthalate, polyurethane poly(hydroxymethyl-p-xylylene-co-p-xylylene) polylactic acid, parylene, fibrin, polytetrafluoroethylene, polyamide, polystyrene, polydimethylsiloxane, polyoxymethylene, Polyacrylonitrile, polytetrafluoroethylene, polycarbonate, polyetheramide, polyvinylidine, polyester, polyethyl cyanoacrilate or polyamine.

In one embodiment, the device comprises a layer of a metal or a metal alloy to which the coating is then applied. In one embodiment, the metal is stainless steel, gold, silver, chromium or titanium. In one embodiment, the metal alloy is silver alloy, titanium alloy, stainless steel alloy or aluminum alloy. In one embodiment, the device comprises a layer of carbon or silica, to which the coating is applied. In one embodiment the device comprises a layer of tungsten, to which the coating is applied.

In one embodiment, the coating comprises a single layer. In another embodiment coating comprises multiple layers, and may comprise any of the materials listed herein. In one embodiment, the layers coating the device are uniform in size and/or content. In one embodiment, the layers differ in size and or content.

In one embodiment, the device is coated by methods known to one skilled in the art. In one embodiment, the device will be spray coated. In one embodiment, the device will be pan coated. In one embodiment, the device will be fluid bed coated. In one embodiment, the device will be spin coated. In one embodiment, the device will be roll coated.

### Device

In one embodiment, this invention provides a device, comprising a coating applied to at least a portion of a surface of the device, wherein the coating comprises any embodiment as herein described.

In one embodiment, this invention provides a device having a coating on at least a portion of a surface of said device, said coating comprising a lipid or phospholipid moiety bound to a polypyranose. In another embodiment, the polypyranose is glycosaminoglycan. In another embodiment the polypyranose is hydroxyethylstarch (HES). In another embodiment, the polypyranose is alginate, In another embodiment, the polypyranose is dextran.

In another embodiment, this invention provides a device having a coating on at least a portion of a surface of said device, said coating comprising a lipid or phospholipid moiety bound to a polygeline (haemaccel).

In one embodiment, this invention provides a device wherein said coating comprises a compound represented by the structure of the general formula (A):

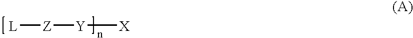

wherein
L is a lipid or a phospholipid;
Z is either nothing, ethanolamine, serine, inositol, choline, phosphate, or glycerol;
Y is either nothing or a spacer group ranging in length from 2 to 30 atoms;
X is a glycosaminoglycan; and
n is a number from 1 to 1000;
wherein any bond between L, Z, Y and X is either an amide or an esteric bond.

In another embodiment, X is polygeline (haemaccel). In another embodiment X is carboxymethylcellulose. In another embodiment X is hydroxyethylstarch (HES). In another embodiment, X is alginate, In another embodiment X is dextran.

In one embodiment, the coated device comprises a cross-linked polymer. In one embodiment, the polymer is cross-linked by using a cross-linking compound or using other cross-linking methods such as UV cross-linking. In one embodiment, the device finds use in medical procedures, for which the device is appropriate. In one embodiment, the devices of this invention prevent, ameliorate or treat vessel blockade or occlusion. In one embodiment of this invention, the device is a stent or a catheter. In another embodiment, the device is an implant. In one embodiment, the device is a dental implant or an orthopedic implant. In one embodiment, the device is an implant for controlled drug delivery. In one embodiment, the device is a bone fixation pin. In one embodiment, the device comprises fixation plates or fixation bolts. It is to be understood, that any device, in particular, any device or implement, with a coating as described herein, for any use, as will be appreciated by one skilled in the art, is to be construed as an embodiment of this invention.

In one embodiment, the device of the present invention is a stent. In one embodiment the stent of the present invention is a slender thread, rod, or catheter placed within the lumen of tubular structures to provide support. In another embodiment, a stent is a device that is used to maintain a bodily orifice or cavity during grafting, or to immobilize a graft following placement. In one embodiment, a stent graft is an intraluminal device that consists of a supporting metal framework and synthetic graft material that is either self-expanding or balloon-expandable. In some embodiments, stent grafts are in three basic configurations, including tube, bifurcated, and aorta-unilateral designs. In some embodiments, stents comprise a modular zigzag structure.

In one embodiment, the stent is an expandable wire mesh that is inserted into a hollow structure of the body to keep it open. In one embodiment, the stent is an expandable hollow perforated tube that is inserted into a hollow structure of the body to keep it open. In one embodiment, the stent is in conjunction with a dilation balloon. In one embodiment, the stent is self-expanding, and thus can dilate or support a blocked conduit in the human body. In one embodiment, the stent is a drug-eluting stents. In one embodiment, the stent continues to release the drugs for a period of up to 60 days after placement.

In one embodiment a stent is fabricated from thin-walled stainless-steel alloy tubes. In one embodiment, a stent comprises intricate patterns in wall openings. In one embodiment, the stent is made of a polymeric material. In some embodiments, the polymer is entirely bioabsorbable. In one embodiment, the stent is made of gold for improved flexibility. In other embodiment the stent is made of a plastic material, polyurethane or silicone.

In one embodiment, the device of the present invention is a catheter. In one embodiment, the catheter of the present invention is a hollow flexible tube for insertion into a body cavity, duct, or vessel. In one embodiment, the catheter is a central venous catheter. In one embodiment, the catheter is a Foley catheter. In one embodiment, the Foley catheter comprises a balloon. In one embodiment, the balloon is coated with a compound of this invention, as well as other desired drugs. In another embodiment, the catheter is a Swan-Ganz catheter. In some embodiments, the catheter is made of materials, which support the specified use of the catheter as will be known to one skilled in the art.

In one embodiment, the catheter comprises medical grade silicone rubber. In one embodiment, the catheter comprises polyurethane. In one embodiment, the catheter comprises teflon. In some embodiments, catheters comprise nylon, dacron, latex

In one embodiment, the catheter comprises a disc mesh/suture flange structure.

In one embodiment, the catheter comprises perfusion holes, dacron felt cuff or retention beads. In some embodiments, the catheter comprises a needle.

In one embodiment, the device of the present invention is a dental implant. In one embodiment, the dental implant is an artificial tooth or bridge that is anchored in the gums or jawbone to replace a missing tooth. In one embodiment, the dental implant is a metal, root-shaped device that is placed surgically in the jawbone. In one embodiment, the dental implant is osseointegrated implant.

In one embodiment, the device of the present invention is an orthopedic implant. In one embodiment, the orthopedic implant is a stem implant. In one embodiment, the stem implant is a hip joint replacement device. In one embodiment, this device includes an elongate curved stem which is adapted for receipt in a cavity formed in the proximal region of a femur, and a spherical head carried on a neck at the upper end of the stem.

In one some embodiments, the medical device comprises a gauze or a sponge. In one embodiment the device is a gauze sponge. In one embodiment, the sponge is a laparotomy or a lap sponge. In one embodiment, the device of the present invention is bandage or a swab.

It is to be understood that the coating of the device will maintain the activities of the compounds of the current invention as will be known to one skilled in the art.

### Methods of Use of the Devices of this Invention

In one embodiment, this invention provides a coated device, wherein the coating comprises any embodiment as herein described. In one embodiment, this invention enables the use of the coated device as described herein in medical applications.

In one embodiment, the coating on the device will suppress and/or inhibit and/or prevent and/or treat atherosclerosis induced coronary artery disease. In one embodiment, the coating on the device will suppress and/or inhibit and/or prevent and/or treat atherosclerosis induced cerebro-vascular disease. In one embodiment, the coating on the device will suppress and/or inhibit and/or prevent and/or treat atherosclerosis induced transient ischemic attack. In one embodiment, the coating on the device will suppress and/or inhibit and/or prevent and/or treat atherosclerosis induced peripheral arterial disease. In one embodiment, the coating on the device will suppress and/or inhibit and/or prevent and/or treat atherosclerosis induced erectile dysfunction. In one embodiment, the coating on the device will suppress and/or inhibit and/or prevent and/or treat inflammation. In one embodiment, the coating on the device will suppress and/or inhibit and/or prevent proliferation. In one embodiment, the coating on the device will suppress and/or inhibit and/or prevent and/or treat cell adhesion.

In one embodiment this invention provides a method of preventing, inhibiting or treating vessel damage or vessel occlusion in a subject comprising the step of applying to said vessel a device having a coating on at least a portion of a surface of said device, said coating comprising a lipid or phospholipid moiety bound to a polypyranose. In another embodiment, the polypyranose is carboxymethylcellulose. In another embodiment, the polypyranose is glycosaminoglycan. In another embodiment the polypyranose is hydroxyethylstarch (HES). In another embodiment, the polypyranose is alginate, In another embodiment, the polypyranose is dextran.

In another embodiment, the coating comprising a lipid or phospholipid moiety bound to polygeline (haemaccel).

In one embodiment, a coated stent of the present invention is used in the oesophageus. In one embodiment, a coated stent of the present invention is used in the trachea. In one embodiment, a coated stent of the present invention is used in the cardiovascular, urinary, urogenital or bilary system.

In one embodiment, percutaneous delivery of the stent is made possible by compacting the device onto a catheter or compressing it into a sheath. In one embodiment, stent implantation is applied to pulmonary arterial stenoses, coarctation, pulmonary and systemic venous obstruction, and obstructed homografts and conduits.

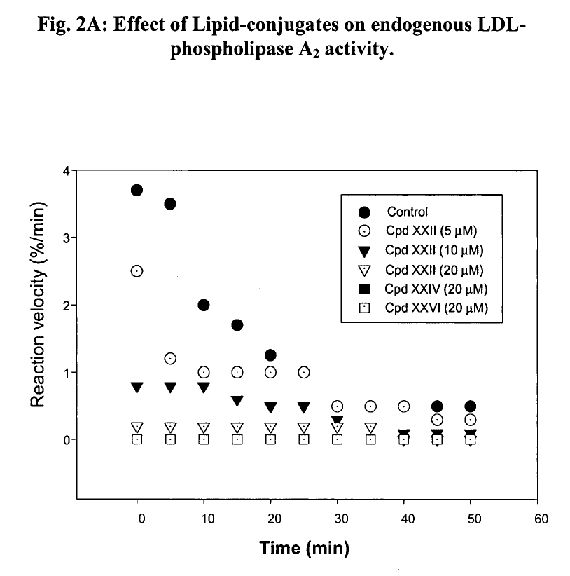

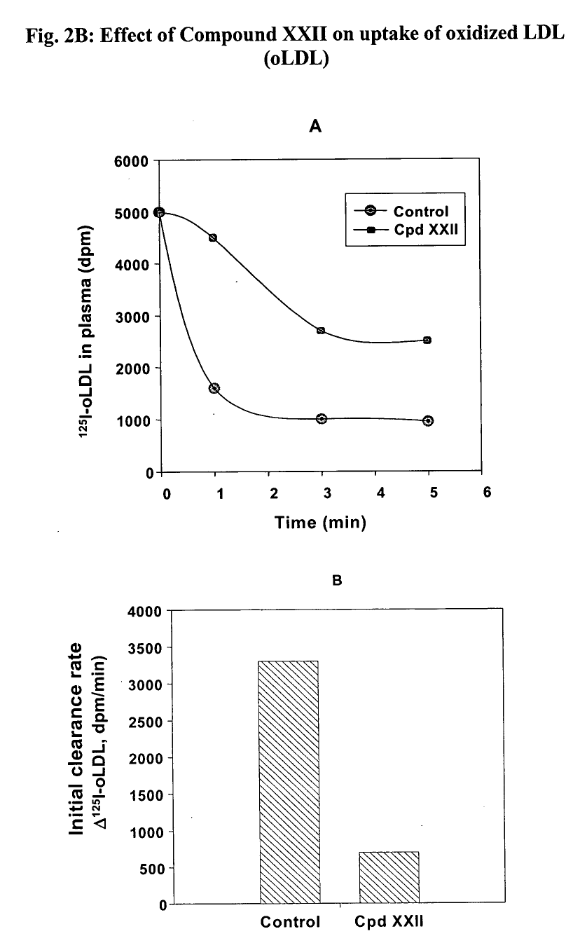

In one embodiment, endovascular stents maintain both arterial and venous patency. In one embodiment, a stent is used to hold open an artery that has become too narrow due to atherosclerosis. In one embodiment, the stent is used for creating an AV fistula. In one embodiment, the coated stents of the current invention are used for the treatment of blocked arteries due to peripheral artery disease. A prominent feature in the pathogenesis of atherosclerosis is the accumulation of blood lipoproteins, such as oxidized low density lipoprotein. In some embodiments, the coating of the stents of the current invention is used for inhibition of oxidized LDL uptake by macrophages as shown in FIGS. 2B and 2B. In some embodiment, the coated stents of the current invention are used for the treatment of renal vascular hypertension treat, hemodialysis access maintenance, Carotid artery disease or Coronary artery disease.

In one embodiment, a coated stent is used in the digestive system. In one embodiment, a coated stent is used in the intestine. In one embodiment, the stent is used for reattaching the intestines after a temporary colostomy. In one embodiment, the stent is used to relieve obstruction in the colon.

In one embodiment, a coated stent is delivered to the ureter to hold it open so the kidney could drain properly. In one embodiment, ureteral coated stent is placed to bypass ureteral obstruction on a long-term basis (months to years) or short term basis (weeks to months). In one embodiment, Short-term stenting may be used as an adjunct to open surgical procedures of the urinary tract to provide a mold around which healing can occur, or to divert the urinary flow away from areas of leakage.

In one embodiment, a coated stent is used in the respiratory system. In one embodiment a coated laryngeal or tracheal stents are used. In one embodiment, coated stents are used as primary treatment for lumen collapse or to stabilize a reconstructive effort of the larynx or trachea to prevent collapse. In one embodiment, coated stents can be used for the larynx and the trachea individually, or they can be used interchangeably or concomitantly.

In one embodiment, bile duct coated stents are used for overcoming obstructions of the bile duct. In one embodiment, the coated stent is about as thick as a ball-point pen refill is used to clear a passage through the bile duct to allow the bile to drain away. In one embodiment, a coated stent will be inserted to a bile duct as part of an endoscopic retrograde cholangio-pancreatography.

In one embodiment, the coated catheter allows the passage of fluids or distends a passageway. In one embodiment, a central venous catheter allows concentrated solutions to be infused with less risk of complications. In another embodiment, central venous catheter permits monitoring of special blood pressures including the central venous pressure, the pulmonary artery pressure, and the pulmonary capillary wedge pressures. In one embodiment, a central venous catheter can be used for the estimation of cardiac output and vascular resistance. In one embodiment, the near end of the catheter may also be connected to a chamber for injections given over periods. In different embodiments, venous catheters may be inserted for the short term or long term.

In one embodiment, the Foley coated catheter has a balloon on the bladder end.

In one embodiment, the Foley catheter is inserted in the bladder, the balloon is inflated (with air or fluid) so that the catheter cannot pull out but is retained in the bladder. In one embodiment, the balloon is coated thus drug is delivered to the vessel wall immediately, in one dose from the balloon.

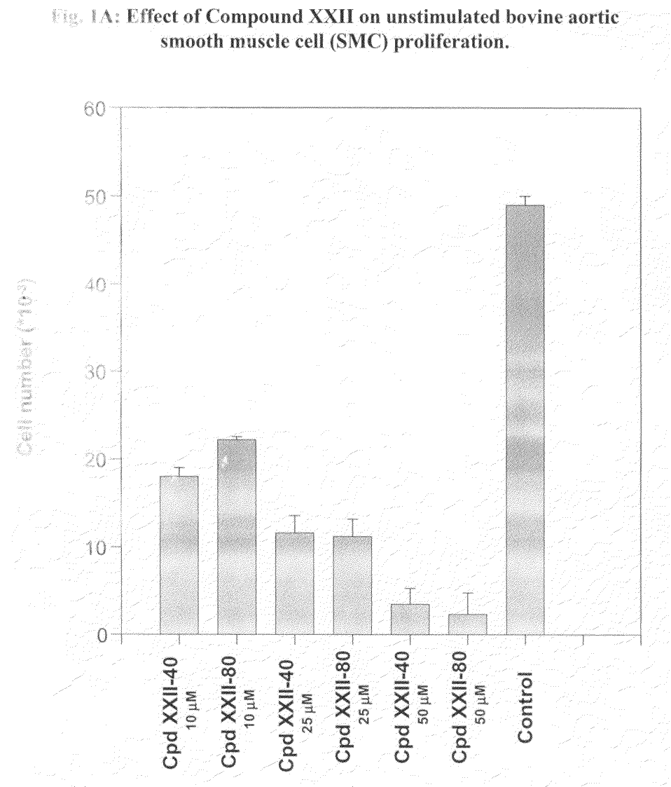

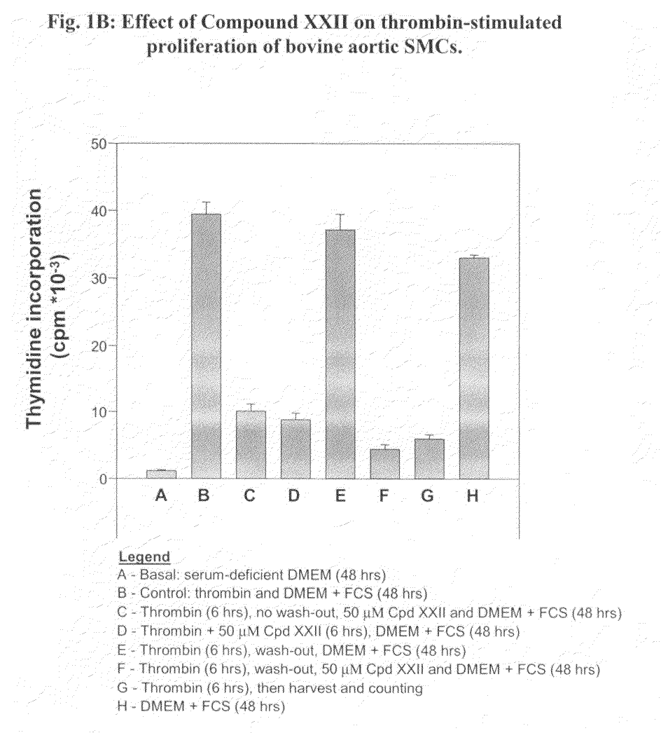

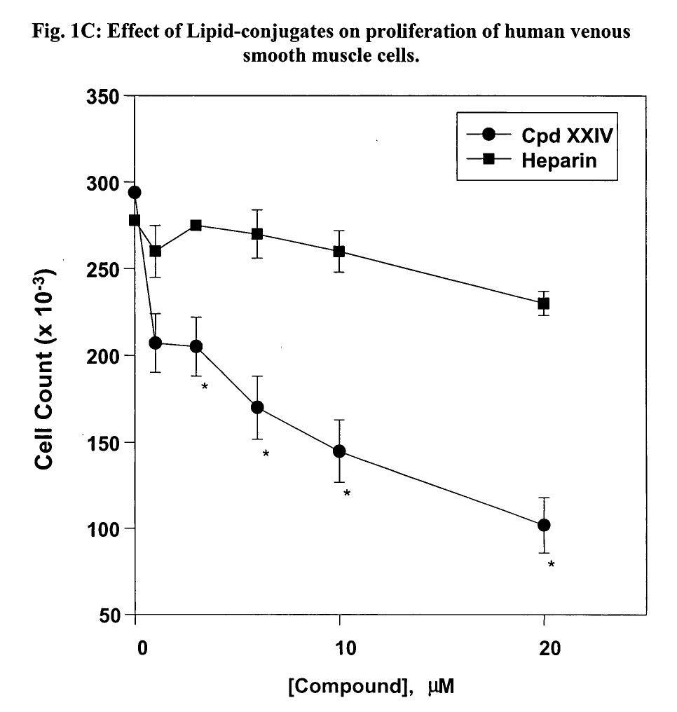

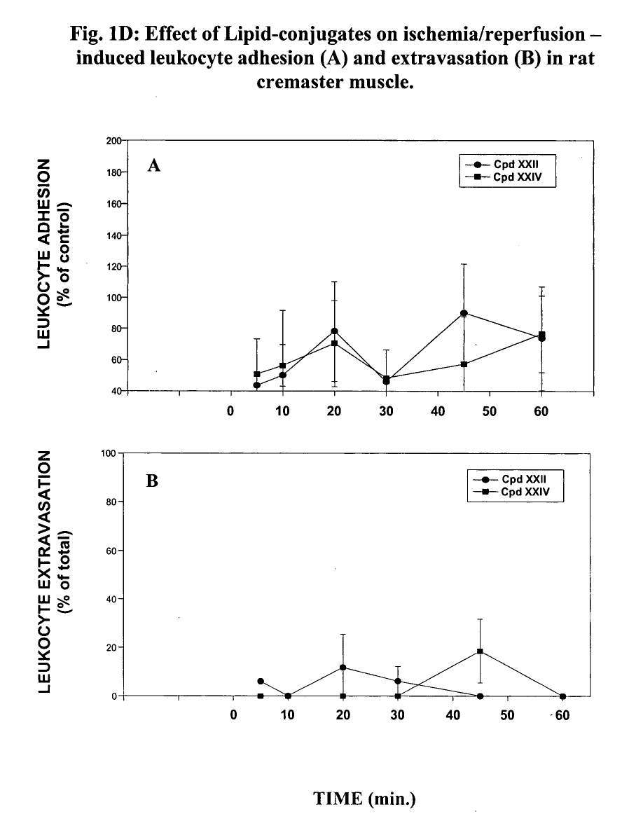

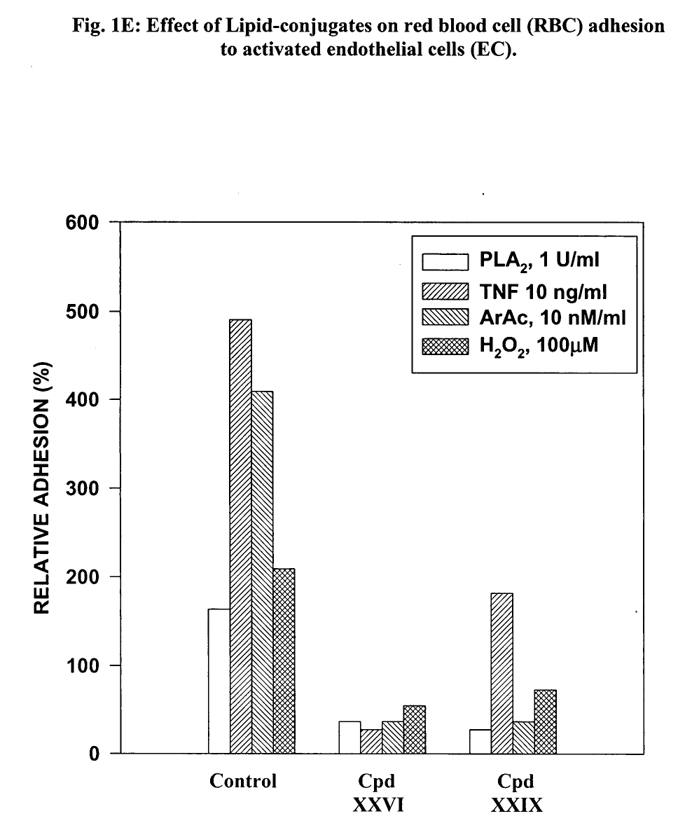

In one embodiment, the Swan-Ganz coated catheter is inserted through the inferior or superior vena cava. In one embodiment, the Swan-Ganz catheter is flow-directed. In one embodiment, the Swan-Ganz catheter utilizes a balloon to direct it to the heart. In one embodiment, the coating possesses anti-proliferative effect. For example, FIG. 1 demonstrated the anti-proliferative effects of the Lipid-conjugates on bovine aortic smooth muscle cells. In one embodiment, the Swan-Ganz catheter is used for measuring a pressure called the pulmonary wedge pressure in front of the temporarily inflated and wedged balloon.

In one embodiment, the compounds of this invention possesses anti-proliferative effect of smooth muscle cells (SMC) wherein fetal bovine serum (FBS) is added and/or a mixture of interleukin 1 (IL-1), fetal bovine serum (FBS) and platelet derived growth factor (PDGF) is added.

In some embodiments administration of Lipid-conjugates has both prophylactic and acute therapeutic benefits when administered in the course of invasive arterial procedures. In one embodiment, the lipid conjugates are useful during balloon angioplasty, as exemplified hereinbelow in Example 1, see Table 2.

In one embodiment, a coated dental implant is used to replace one or more teeth without affecting bordering teeth. In another embodiment, a coated dental implant is used to support a bridge and eliminate the need for a removable partial denture. In one embodiment, a coated dental implant is used to Provide support for a denture, making it more secure and comfortable. In some embodiments, the coated dental implant is endosteal or subperiosteal.

In one embodiment, coated bone fixation pins, nails, screws, or plates are used for external fixation which involves the use of these assemblies through the bone attached to a steel rod outside the limb. In one embodiment, external fixation is used primarily to stabilize transverse fractures.

In one embodiment, gauze, sponge, bandage or a swab is used in treating injuries. In some embodiments, these medical devices are used during surgery. In one embodiment, these devices are used during the healing process of a wound. Other embodiments, in which make use of these devices will be known to one skilled in the art.

In one embodiment, the devices of the present invention are used to treat a medical condition, which arises as a result of, or is further complicated by an overproduction of cytokines. In some embodiments, the application of the device itself stimulates overproduction of cytokines and in some embodiments, subsequent tissue damage, or in another embodiment, obstruction or occlusion, or cellular overgrowth on the device, rendering the device less efficient, or in another embodiment, ineffective. In some embodiments, coating of the device as described herein, prevents, or mitigates.

For example, and in some embodiments, tumor necrosis factor (TNF)-alpha levels are raised in subjects in which a device is implanted, and, in one embodiment, the coating of the device with the compounds, as herein described, reduce levels of TNF in the subject.

In one embodiment, the devices for use according to the methods of the present invention treat or ameliorate oxidative injury, which in one embodiment, can be caused or exacerbated by microbial infections such as those that can be caused by the medical procedures utilizing the coated device of the current invention.

Administration of the Lipid-conjugates in a diversity of animal and cell models of disease invoked remarkable, and unexpected, cytoprotective effects, which, as exemplified herein, are useful in the treatment of diseases related to pathogenic infection that can be caused by medical procedures utilizing the coated device of the present invention. For example, the lipid-conjugates as exemplified herein, increased survival of septic rats, reduced TNF-α and IL-6 mRNA and protein levels, reduced sPLA2-IIA and iNOS mRNA, and reduced ICAM-1 protein in cell and animal models of sepsis (as exemplified in U.S. application Ser. No. 10/627,981, U.S. application Ser. No. 10/919,523, and U.S. application Ser. No. 10/952,496, and other Applications referenced therein, all of which are incorporated herein by reference in their entirety) and dose-dependently inhibited PLA₂ enzyme activity.

In one embodiment, the compounds for the use in the present invention also reduce sPLA2 expression. Experiment 4.1 demonstrates the profound anti-inflammatory effect of compound XXII on the inhibition of PLA₂ enzyme. Other inflammatory mediators, for example, as described in U.S. application Ser. No. 10/952,496, which is incorporated by reference herein.

In one embodiment, the compounds for the use in the present invention also reduce MCP-1 expression. MCP-1 has been found in the joints of people with rheumatoid arthritis where may serve to recruit macrophages and perpetuate the inflammation in the joints. MCP-1 has also been found elevated in the urine of people with lupus as a sign warning of inflammation of the kidney.

In one embodiment, the compounds for the use in the present invention also reduce TNF expression. TNF is a cytokine involved in systemic inflammation and is a member of a group of cytokines that all stimulate the acute phase reaction. TNF causes apoptotic cell death, cellular proliferation, differentiation, inflammation, tumourigenesis, and viral replication.

In one embodiment, the compounds for the use in the present invention also reduce cell adhesion. Cell adhesion may be indication of inflammation. The compounds for the use in the present invention reduce adhesion of U937-SMC cells.

### Methods of coating the Devices of this Invention

In one embodiment a coating solution is first prepared by dissolving or dispersing a pH buffer agent, the polymer, and the cross-linking compound, as well as a bioactive agent and the like, if any, in a solvent. In some embodiments, the solvent can be an organic solvent, an aqueous solvent or a mixture of two or more solvents. In one embodiment, the solution is then applied onto a surface of the device. In different embodiments the solution can be applied by dipping, spraying, or painting. In one embodiment, cross-linking of the polymer takes place either when the solvent is present in the coating or after the solvent has been removed from the coating. In one embodiment, the coating solution may be prepared by dissolving the buffer and the cross-linking compound in the polymer without using a solvent. In one embodiment, the polymer is cross-linked after the solution has been applied onto a surface of a support member.

### EXAMPLES

The abbreviations used in the examples below are:

PE=phosphatidyl-ethanolamine
HA=hyaluronic acid

### Cpd=Compound

Compound XXII=PE conjugated to HA
Compound XXIII=dimyristoyl-phosphatidyl-ethanolamine linked to HA
Compound XXIV=PE conjugated to heparin
Compound XXV=PE conjugated to chondroitin sulfate A (CSA)
Compound XXVI=PE conjugated to carboxymethyl cellulose (CMC)
Compound XXVII=PE conjugated to Polygeline (haemaccel)
The compounds used in the examples below were prepared as described in U.S. patent application Ser. No. 10/952,496, which is incorporated herein by reference.

### Example 1

### Prophylaxis For Invasive Surgical Procedures, Including Catheterization

The Lipid-conjugates are effective in the treatment and prophylaxis for cardiovascular disease in many settings, including atherosclerosis, as described below, as well as in the setting of stenosis and restenosis induced by ischemia/reperfusion injury. The lipid-conjugates are effective in preventing the formation of stenotic lesions as may occur in the course of invasive surgical procedures which involve manipulation of vascular organs, in particular vascular catheterization.

Since the proliferation of vascular smooth muscle cells (SMC) is the process leading to blood vessel stenosis, the Lipid-conjugates were assessed for their effect on this process.

Experiments 1.1-1.3 demonstrate the anti-proliferative effects of the Lipid-conjugates on bovine aortic smooth muscle cells, unstimulated or stimulated by thrombin, and on the proliferation of human venous smooth muscle cells.

Experiment 1.1: For unstimulated cells, bovine aortic smooth muscle cells were seeded at 7×10³ cells per well (in 24-well plates), in DMEM supplemented with 10% fetal calf serum, in the absence or presence of Compound XXII-40 or Compound XXII-80 (enriched with PE), grown for 72 h, and counted in Coulter (FIG. 1A).

Experiment 1.2: For stimulated cells, bovine aortic smooth muscle cells were grown under the conditions as above for 48 h, following pre-incubation for 6 h, as indicated, with either thrombin, DMEM supplemented with fetal calf serum, Lipid-conjugate, or a combination thereof. Cell growth is represented as the amount of thymidine incorporation (FIG. 1B).

Experiment 1.3: SMC from human saphenous vein, were inoculated at 8×10⁴ cells/5 mm culture dish, in DMEM supplemented with 5% fetal calf serum and 5% human serum. A day later the cells were washed and incubated in the same culture medium in the absence (control) or presence of the Lipid-conjugate (Compound XXIV) or its polymeric carrier (heparin, at the same concentration as the Compound XXIV). After 5 days, the cells were harvested (by trypsinization) and counted (FIG. 1C). Each datum is mean ±SEM for 3 replications. Results were reproducible in a repeat experiment. *p<0.005.

Experiment 1.4: Ischemia/reperfusion injury: As noted above, the injury induced by ischemia and reperfusion, is the major stimulant for stenosis subsequent to catheterization, surgery or other procedures that involve vascular obstruction and occlusion. To demonstrate the ability of the Lipid-conjugates to ameliorate this injury, they were tested for inhibition of white cell adhesion and extravasaion, which signal ischemia/reperfusion injury to blood vessels. Leukocytes were labeled in vivo by i.v. injection of rhodamine. Ischemia was applied to exposed cremaster muscle in rats (in situ) for 90 min, then blood flow was restored for reperfusion. The fluorescent-labeled leukocytes adherent to blood vessel walls (FIG. 1D) and those extravasated to the extravascular space (FIG. 1D) were videotaped and counted at the indicated time point during the reperfusion period. Lipid-conjugates (10 mg/100 g body weight) were injected i.v. 40 min and 10 min prior to induction of ischemia. FIG. 1D shows that administration of Lipid-conjugates efficiently suppresses the ischemia/reperfusion-induced adhesion and extravasation of leukocytes. Each datum is mean ±SEM obtained from 5 rats treated with Compound XXII and 3 rats treated with Compound XXIV. p<0.005.

Experiment 1.5: Another expression of damage to blood vessel wall endothelium is adhesion of red blood cells (RBC) to endothelial cells upon their activation by oxygen radicals, lipid mediators or cytokines (produced subsequent to ischemia reperfusion injury). RBC adherence further facilitates vascular occlusion. To demonstrate that the protective effect of Lipid-conjugates on endothelium, bovine aortic endothelial cells were exposed to tumor necrosis factor (TNF-α), phospholipase A₂, arachidonic acid (ArAc), or hydrogen peroxide, and then assayed for cytodamage, as judged by adhesion of red blood cells as an index of endothelial intactness. Bovine aortic endothelial cells (BAEC) were pre-incubated for 30 min with either 5 μM Compound XXVI or 20 μM Compound XXIX, then washed and stimulated for 18 h with TNF, ArAc, or PLA₂ at the indicated concentration. For stimulation with H₂O₂, the cells were treated with H₂O₂ for min, then washed and incubated in the control culture medium for 18 h. The BAEC were washed and incubated with human red blood cells (RBC) for 30 min. The cultures were washed, and the RBC which remained adhering to the BAEC were counted under a microscope (FIG. 1E).

Experiment 1.6: Balloon-induced stenosis in rats: The efficacy of systemic and intra-arterial administration of Lipid-conjugates in protocols for balloon-induced stenosis in the carotid artery of rats were examined (Table 2). Rats were pre-treated with an i.p. injection of 10 mg/100 g body weight of Compound XXII or Compound XXVI in PBS, or PBS alone, 1 day and 1-2 hours before injury. Injury was achieved using the standard Fogarty catheter. The distal left common and external carotid arteries were exposed through a midline incision in the neck. The left common carotid artery was denuded of endothelium by the intraluminal passage of a 2F Fogarty balloon catheter (Baxter, Santa Anna, Calif.) introduced through the external carotid artery. The catheter was passed three times with the balloon distended sufficiently with saline to generate a slight resistance. After injury, the rats were injected with 10 mg/100 g body weight of Compound XXII, Compound XXVI, or vehicle every day for 3 days, and then every other day, for a total of 8 post-injury Lipid-conjugate injections.

For intra-arterial administration, when the catheter was removed after injury, a polyethylene (PE-10) tube connected to a syringe was introduced into the common carotid artery. A segment of the common carotid artery was temporarily isolated by sliding ligature and vascular clamp. Approximately 50 μl of solution containing 10 nmole of Compound XXVI was injected into isolated arterial segment and left in place for 15 min. The drug solution was then evacuated and the external carotid artery was ligated.

Rats were sacrificed on the 14^{th} day, and their arteries were processed according to a standard procedure. Half of the rats were injected with bromodeoxyuridine (BrdU), their arteries fixed with formalin and triton, and processed for BrdU staining. The percent of luminal stenosis (in the damaged area) was determined by histological measurement of neointima (N) to media (M) area ratio (Table 2)

TABLE 2
Inhibition of Balloon-Induced Stenosis
in Rats by Lipid-Conjugates

| Method of | | | | | |
|---|---|---|---|---|---|
| Adminis- | | % stenosis | | | |
| tration | Treatment | (Mean ± SEM) | P | N/M | P |
| i.p | Untreated | 53.96 ± 4.11 | 0.003 | 1.64 ± 0.12 | 0.001 |
| | (n = 7) | | | | |
| | Cpd XXII | 53.96 ± 2.89 | | 1.0 ± 0.08 | |
| | (n = 6) | | | | |
| i.p. | Untreated | 41.53 ± 4.84 | 0.023 | 1.16 ± 0.12 | 0.036 |
| | (n = 6) | | | | |
| | Cpd XXVI | 21.89 ± 5.42 | | 0.64 ± 0.17 | |
| | (n = 8) | | | | |
| Intra- | Untreated | 53.12 ± 12.8 | 0.052 | 1.61 ± 0.17 | 0.008 |
| Arterial | (n = 4) | | | | |
| | Cpd XXVI | 29.64 ± 2.17 | | 0.99 ± 0.08 | |
| | (n = 6) | | | | |

These experiments demonstrate that administration of Lipid-conjugates are effective therapy in the treatment of cardiovascular disease, by a plurality of mechanisms, including inhibition of vascular smooth muscle cell proliferation, uptake of lipoprotein, oxidative stress, and leukocyte activation in models of ischemia and reperfusion. Administration of Lipid-conjugates is of both prophylactic and acute therapeutic benefit when administered in the course of invasive arterial procedures, particularly balloon angioplasty.

### Example 2

### Cardiovascular Disease

The Lipid-conjugates are effective therapy for ischemic vascular disease, atherosclerosis, and reperfusion injury. This is demonstrated in Experiments 2.1-2.3.

A prominent feature in the pathogenesis of atherosclerosis is the accumulation of blood lipoproteins, such as oxidized low density lipoprotein (oLDL), in cells lining vascular walls, and the proliferation of cells lining and within vascular walls, such as smooth muscle cells. The resultant narrowing of the blood vessel lumen at the site of the atherosclerotic lesion may give rise to varying degrees of tissue ischemia. While ischemic events may be reversible, either spontaneously or through medical intervention, the process of tissue injury may persist to the stage of reperfusion injury, in which the previously ischemic tissue is still at risk for damage, through several mechanisms, including oxidative damage.

Experiment 2.1: LDL-PLA₂. Endogenous LDL-phospholipase A₂ (PLA₂) hydrolyzes LDL-phospholipids to form lyso-phospholipids, which are chemotactic and facilitate LDL oxidation and uptake by blood vessel wall cells. In order to demonstrate that the Lipid-conjugates inhibit LDL-associated PLA₂ activity, LDL (0.1 μM) was incubated for 15 min at 37° C. in the absence or presence of Compound XXII, Compound XXIV or Compound XXVI at the concentrations indicated (FIG. 2A). At time 0, C₆-NBD-PC (0.5 μM) was added to the dispersion. This resulted in an instantaneous increase of fluorescence intensity (due to incorporation of NBD into lipidic cores). When LDL was incubated alone the increase of fluorescence was followed by time-dependent decrease of fluorescence intensity that can be attributed to hydrolysis of the LDL-associated PLA (and subsequent departure of the resultant NBD-caproic acid from the LDL particle to the aqueous medium). When LDL was incubated in the presence of Compound XXII, Compound XXIV or Compound XXVI this time-dependent decrease was fully or partially inhibited.

Experiments 2.2-2.3: To demonstrate that the Lipid-conjugates inhibit LDL uptake by cultured macrophages and in whole animals, human LDL (isolated by the conventional method of floatation) were subjected to Cu²⁺-induced oxidation, and labeled with ¹²⁵I. Confluent J774 macrophages were incubated with 100 μM ¹²⁵I-oLDL and Lipid-conjugate at the indicated concentration in PBS buffer (pH=7.4) supplemented with 0.5% BSA, for 3 h. The cells were then washed 4 times with the PBS/BSA, and subjected to lysis by 0.1 N NaOH for 30 min. The cell lysate was collected and the ¹²⁵I content was determined in a radioactivity counter (Table 3).

TABLE 3
Inhibition of Oxidized LDL Uptake in Macrophages
by Compound XXII and Compound XXIV

| | Cell-associated | |
|---|---|---|
| Treatment | ¹²⁵I-oLDL (DPMx 10⁻³) | % Inhibition |
| Control | 92.2 ± 4.0 | |
| 10 μM Compound XXII | 20.9 ± 1.7 | 78% |
| 20 μM Compound XXIV | 59.2 ± 8.3 | 37% |

Experiment 2.3: Uptake of oLDL in-vivo: Rats weighing 200 g were injected i.v. with 0.4 ml saline containing 250 nmole of Cu²⁺-induced oxidized LDL labeled with ¹²⁵I, and 200 mmole of Compound XXII. Blood samples were drawn at the indicated time intervals and the ¹²⁵I radioactivity in the plasma was counted (FIG. 2B). The initial clearance rate was calculated as the change in ¹²⁵I radioactivity in blood samples drawn after one minute compared to ¹²⁵I radioactivity in blood samples at time 0 (FIG. 2B).

These experiments demonstrate that administration of Lipid-conjugates is effective therapy in the treatment of cardiovascular disease, including atherosclerosis. Additional support for the capacity of the Lipid-conjugates to treat cardiovascular diseases is provided in Experiments 7.1-7.3 and Experiments 9.3 below, showing that the Lipid-conjugates inhibit proliferation of smooth muscle cells, and protect LDL from oxidative damage.

### Example 3

### Anti-Oxidant Therapy

The noxious effect of peroxide free radicals on living tissue is known as oxidative damage. When cell membranes are the targets for this damaging process, membrane dysfunction and instability result. Oxidative damage to blood proteins, particularly blood lipid proteins, results in their over-accumulation in cells lining the vasculature, thus contributing to atherogenesis. In fact, oxidative cell damage is a major mechanism attributed to the process of aging or senescence.

In order to determine the effect of Lipid-conjugates on oxidative damage to proteins or cell membranes, tissue was exposed to hydrogen peroxide (H₂O₂) produced by (a) the enzyme glucose oxidase (GO) in the absence or presence of additional membrane destabilizing agents such as PLA₂ or (b) by exposure to divalent cations, such as copper.

Experiments 3.1-3.3 demonstrate the ability of Lipid-conjugates to preserve cells from oxidative damage, as judged by the cells' retention of both arachidonic acid and of low molecular weight intracellular substances.

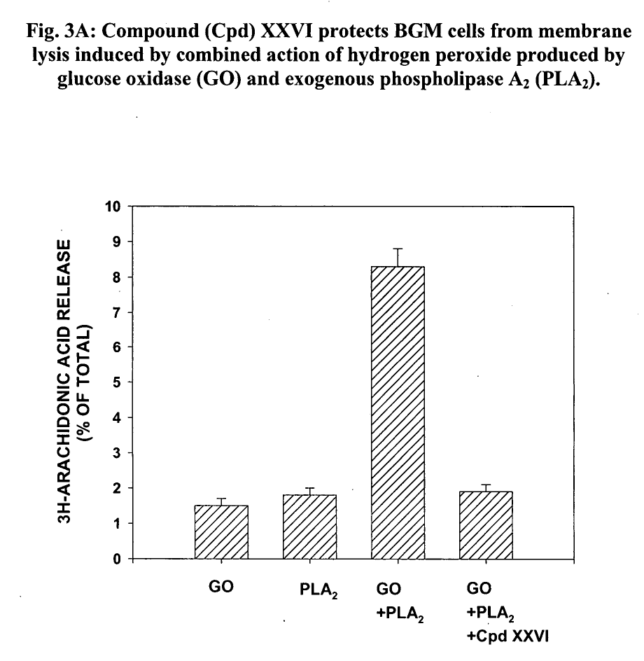

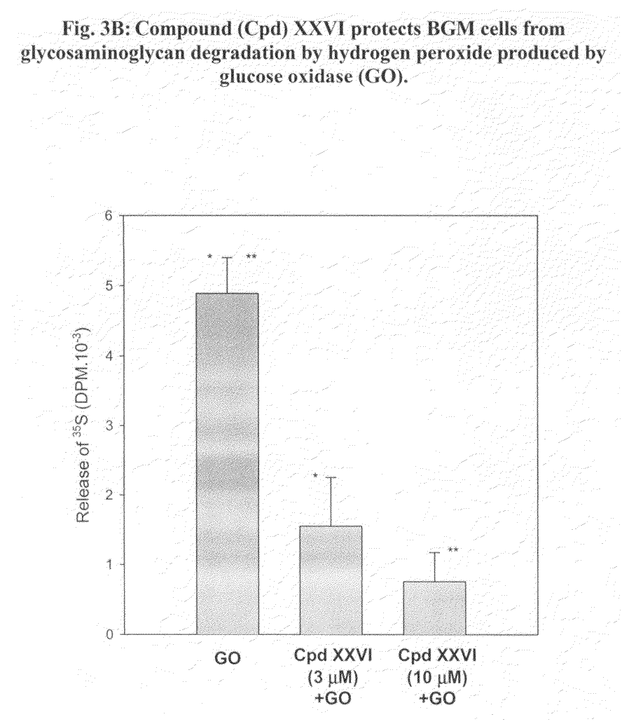

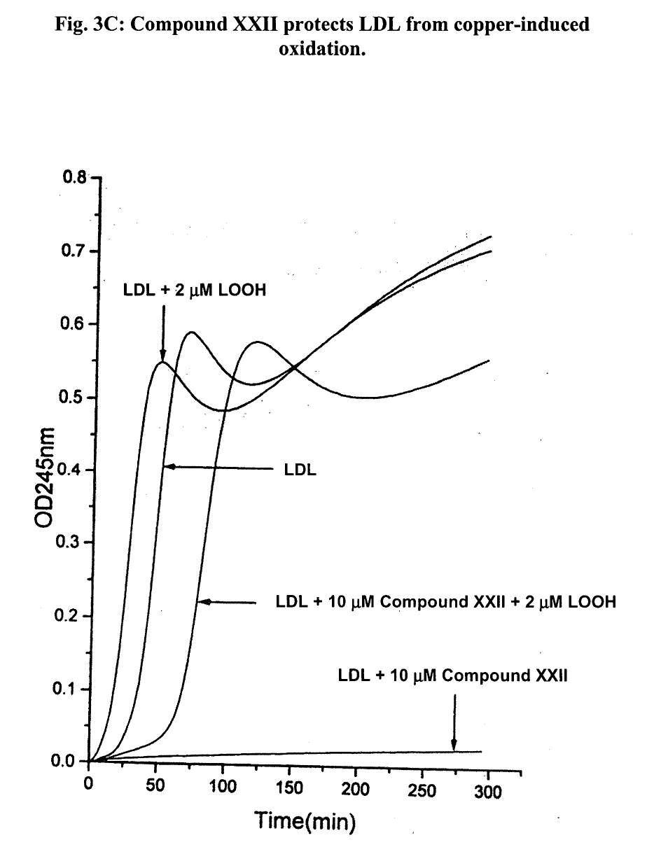

Experiment 3.1: Confluent BGM (green monkey kidney epithelial) cells were labeled with ³H-arachidonic acid. The cells were treated with Compound XXVI for 30 min prior to treatment with GO (an H₂O₂ generator) and PLA₂ (0.5 U/ml) (FIG. 3A).

Experiment 3.2: BGM cells were labeled with ³⁵SO₄ overnight. The cells were washed with DMEM (containing 10 mg/ml BSA) 4 times with PBS. The cells were then incubated in DMEM supplemented with GO for 90 min, and the culture medium was collected and counted for ³⁵S radioactivity. For treatment with Compound XXVI, cells were incubated with 3 or 10 μM Compound XXVI for 30 min prior to introduction of GO. Data are presented as mean ±SEM for 5 replications. *p<0.005; **p<0.001 (FIG. 3B).

Experiment 3.3 demonstrates the ability of Lipid-conjugates to inhibit the oxidation of blood lipoprotein. Low density lipoprotein (LDL; 0.1 μM) and or hydroperoxides (LOOH) were incubated in the absence and presence of various concentrations of Compound XXII or hyaluronic acid at 37° C. At time 0, 5 μM CuCl₂ was added to the dispersions, and the mixtures were continuously monitored for oxidation products at 245 nm (FIG. 3C). The absorbance at 245 (OD units) is depicted as a function of time.

These experiments demonstrate that administration of Lipid-conjugates is an effective therapy to prevent tissue damage induced by oxidative stress (associated with free radical and hydrogen peroxide production) by a plurality of mechanisms, including inhibiting the oxidation of lipoprotein, inhibiting arachidonic acid release, and preserving the integrity of cell membranes (inhibiting GAG degradation), including red blood cell membranes, as described above. The efficacy of Lipid-conjugates in protecting against tissue damage induced by oxidative stress may contribute to their usefulness in treating conjunctivitis.

### Example 4

### PLA₂ Inhibition

Experiment 4.1. The PLA₂ enzymes catalyze the hydrolysis of fatty acids attached to phospholipids on the plasma membrane. Arachidonic acid, the main metabolite released from these reactions, is a precursor for other enzymatic reactions mediated by lipoxygenases and cyclooxygenases. These reactions produce prostaglandins and leukotrienes, which have a profound effect on inflammation in vivo. Therefore, PLA₂ inhibitors are capable of inhibiting inflammation via their ability to inhibit the production of downstream inflammatory factors.

Experiments were designed to determine the effect of Compound XXII, Compound XXV, Compound XXX, and Compound LXXXVIII on the inhibition of the Naja Naja Snake Venom PLA₂ enzyme in an in vitro fluorometric assay. The reaction of the PLA₂ enzyme and the PLA₂ enzyme substrate 2-(6-(7-nitrobenz-2-oxa-1,3diazol-4-yl)amino) hexanoyl-1-hexadecanoyl-sn-glycero-3-phosphocholine (NHGP) yields a product, which can be detected using a fluorometer. Decreased absorbance indicates inhibition of the PLA₂ enzyme.

### Methods

Compound XXII and Compound XXV were solubilized and diluted in D-PBS, and tested at final concentrations of 0.625, 0.125, 0.25, 0.5 and 1 mg/ml. Compound XXX and Compound LXXXVIII were solubilized in 100% dimethyl sulfoxide (DMSO), diluted in D-PBS and tested at final concentrations of 0.01, 0.1 and 1 mg/ml. 1 mM NHGP was diluted in D-PBS, for a final concentration of 1 μM. The positive control, Mefenamic Acid (Sigma, M-4267), was tested at a final concentration of 0.1 mg/ml. The PLA₂ enzyme is derived from the Naja Naja Snake Venom (Sigma, P6139) and tested at a final concentration of 5 Units/ml. The reaction was carried out in 200 μl solution and initiated by addition of substrate. Fluorescence was read immediately and then every minute for 30 minutes for a total of 30 readings. The fluorometer was set as follows: Excitation 450/50; Emission 530/25; Gain 50.

### Results

Compound XXII inhibited the PLA₂ enzyme by 37%, 42%, 71% and 98% at 0.125, 0.25, 0.5 and 1 mg/ml respectively compared to 41% inhibition by 0.1 mg/ml mefenamic acid, which served as a positive control. Compound XXV inhibited the PLA₂ enzyme, although with no apparent dose response, by 20%, 30% and 26% at 0.625, 0.125, 0.25 mg/ml. The inhibition of the PLA₂ enzyme by Compound LXXXVIII and Compound XXX could not be determined in this assay, due to difficulties in solubilizing the compounds in DMSO, even after sonication.

Thus, Compound XXII inhibits the PLA₂ enzyme in a dose-dependent manner, indicating its ability to act as an anti-inflammatory drug. Other experiments showing anti-inflammatory effects of Compound XXII are demonstrated in U.S. application Ser. No. 10/989,607, filed Nov. 17, 2004 and are hereby incorporated by reference.

Other examples in which Lipid-conjugates were effective in treating diseases such as obstructive respiratory disease, intestinal diseases, multiple sclerosis, skin diseases, cardiovascular disease, prophylaxis for invasive surgical procedures, invasive cellular proliferative disorders, lung injury, transplant organ rejection, etc can be found in U.S. application Ser. No. 10/627,981, U.S. application Ser. No. 10/919,523, and U.S. application Ser. No. 10/952,496, which are incorporated herein by reference in their entirety.

### Example 5

### Anti-Inflammatory Effects of the Lipid Conjugates on Smooth Muscle Cells (SMC)

### Methods:

A solution of about 20 mg/ml of lipid conjugate (whose synthesis is as described in U.S. patent application Ser. No. 10/952,496, which is incorporated herein by reference, and/or as further described hereinunder) was prepared by mixing the dry material in the buffer, the solution was vortexed thoroughly, preferably with warming, and then sonicated in a bath, using a cap-horn sonicator to get a clearer more homogenous suspension. Alternatively, the suspension was stirred on a warm plate (up to 50° C.) until a homogenous, almost clear suspension was obtained. Diluted solutions can be filtered for sterilization.

Human coronary artery smooth muscle cells (HCASMC) purchased from ATCC were cultured with 2 mg/ml of the lipid conjugates (corresponding to about 40 μM).

### Results:

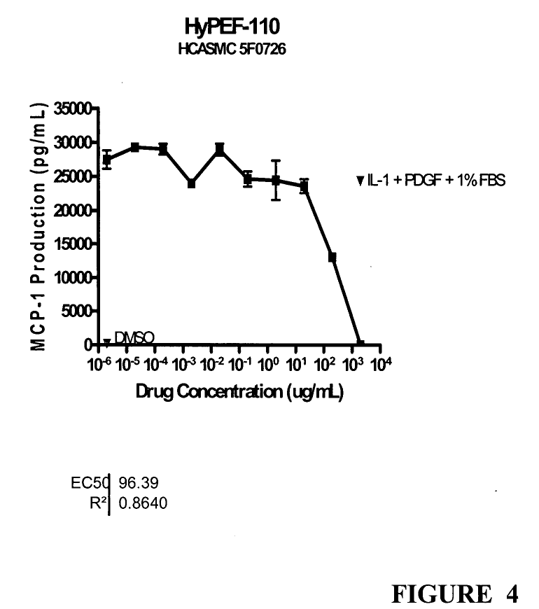

The IC₅₀ (the half maximal inhibitory concentration) of the indicated lipid conjugates on MCP-1 production by HCASMC was assessed, whose production is an indicator of inflammation. Cells were incubated with medium and 1% fetal bovine serum (FBS), with and without interleukin-1 (IL-1) and platelet derived growth factor (PDGF). Representative compounds of this invention were utilized in this context. IC50 results are presented in Table 4. Representative graph depicting the effect of lipid conjugates on smooth muscle cells (SMC) is shown in FIG. 4.

TABLE 4

| compound | IC50 (μg/mL) | IC₅₀ (nM) | % inhibition |
|---|---|---|---|
| XXIIf-110 | 96 | 1928 | 100 |
| XXIIt-110 | 8 ± 7 | 155 ± 134 | 82 |
| XXIII-120 | 108 ± 75 | 2150 ± 1513 | 91 |
| XXIV-130 | 17 | 341 | 100 |
| XXV-75 | 29 | 580 | 82 |
| XXV-100 | 14 ± 17 | 290 ± 325 | 98 |
| LI-120 | 8 | 160 | 60 |
| HemPE80 | Not significant | Not significant | |
| XXVIII90 | Not significant | Not significant | |
| XLV 40/100 | Not significant | Not significant | |
| XLV 10/60 | Not significant | Not significant | |
| XLV 5/60 | 66 | 1320 | 50 |

### Example 6

### Anti-Inflammatory Effects of the Lipid Conjugates on U937 Cells

### Methods:

Lipid conjugates were prepared as described in Example 5. U937 cells purchased from ATCC were cultured with 2 mg/ml of the lipid conjugates (corresponding to about is 40 μM)

### Results:

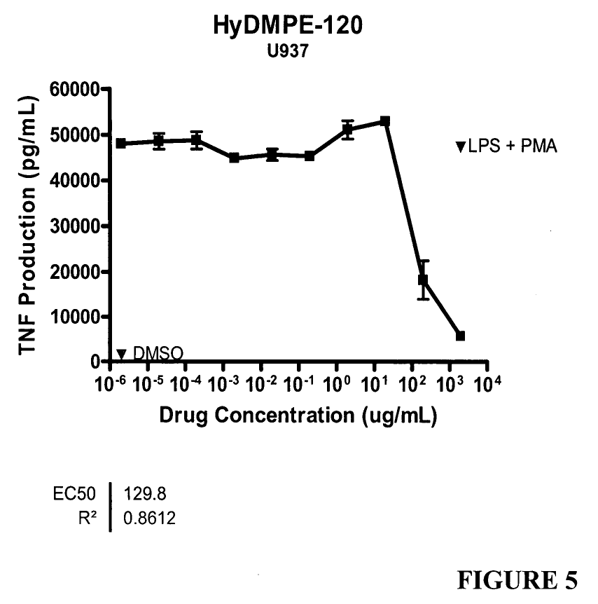

The IC₅₀ values of lipid conjugates on U937 production of TNF were obtained, and taken to reflect inflammation. U937 cells were cultured with the lipid conjugates in increasing cocentration in the presence of the inflammatory stimmuli lipopolysaccharide (LPS) and phorbol 12-myristate 13-acetate (PMA), and the IC₅₀ was determined. Representative compounds of this invention were utilized in this context and the results are presented in Table 5. Representative graph depicting the effect of lipid conjugates on U937 cells is shown in FIG. 5.

TABLE 5

| compound | IC₅₀ (μg/mL) | IC₅₀ (nM) | % inhibition |
|---|---|---|---|
| XXIIf-110 | 84 ± 109 | 1673 ± 2179 | 95 |
| XXIIt-110 | 69 ± 37 | 1374 ± 745 | 98 |
| XXIII-120 | 133 ± 5 | 2663 ± 95 | 88 |
| XXIV-130 | 77 ± 12 | 1540 ± 236 | 50 |
| XXV-75 | 70 ± 55 | 1392 ± 1095 | 96 |
| XXV-100 | 22 ± 9 | 443 ± 171 | 71 |
| LI-120 | Not significant | Not significant | |
| HemPE80 | Not significant | Not significant | |
| XXVIII90 | 989 | 19778 | 100 |
| XLV 40/100 | Not significant | Not significant | |
| XLV 10/60 | Not significant | Not significant | |
| XLV 5/60 | Not significant | Not significant | |

### Example 7

### Lipid Conjugate inhibition of U937 adhesion to SMC Cells

### Methods:

The lipid conjugates were prepared as described in Example 5.

Co-cultures of U937 and HCASMC (5F0726) cells were prepared, and the indicated conjugates were added to the cultures at increasing concentrations, in the presence of LPS and PMA. Average adherence ratios were deduced.

### Results:

The average adherence ratio was assessed as a function of lipid conjugate concentration in the presence of lipopolysaccharide (LPS) and phorbol 12-myristate 13-acetate (PMA) were taken as a measurement of inhibition of inflammation. Representative compounds of this invention were utilized in this context. Results are presented in Table 6. Representative graph depicting the effect of lipid conjugates on adherence of U937 cells to smooth muscle cells is shown in FIG. 6.

TABLE 6

| compound | IC₅₀ (μg/mL) | IC₅₀ (nM) | % inhibition |
|---|---|---|---|
| XXIIf-110 | 457 ± 99 | 9136 ± 1988 | 95 |
| XXIIt-110 | 361 ± 129 | 7218 ± 2571 | 98 |
| XXIII-120 | 419 ± 45 | 8390 ± 891 | 88 |
| XXIV-130 | Not significant | Not significant | |
| XXV-75 | 442 | 8840 | 44 |
| XXV-100 | Not significant | Not significant | |
| LI-120 | 517 | 10340 | 41 |
| HemPE80 | Not significant | Not significant | |
| XXVIII90 | Not significant | Not significant | |
| XLV 40/100 | Not significant | Not significant | |
| XLV 10/60 | Not significant | Not significant | |
| XLV 5/60 | Not significant | Not significant | |

### Example 8

### Anti-Proliferative Effects of the Lipid Conjugates in Smooth Muscle Cells (SMC)

### Methods:

The lipid conjugates was prepared as described in Example 5.

Cell proliferation of HCASMC (5F0726) in the presence of the lipid conjugates at increasing concentration was assessed when cells were cultured in the presence of 1% FBS with or without IL-1 and PDGF.

### Results:

Changes in cell fluorescence was taken as a measure of diminished proliferation, with results presented in Table 7. Representative graphs depicting the effect of lipid conjugates on proliferation of smooth muscle cells (SMC) is shown in FIG. 7A and when cultured with interleukin-1 (IL-1), platelet derived growth and factor (PDGF) is shown in FIG. 7B.

TABLE 7

| | SMC (FBS) | | | SMC (IL-1 + PDGF + FBS) | | |
|---|---|---|---|---|---|---|
| | IC₅₀ | | | IC₅₀ | | |
| compound | (μg/mL) | IC₅₀ (nM) | Inhibition % | (μg/mL) | IC₅₀ (nM) | Inhibition % |
| XXIIf-110 | 239 ± 147 | 4783 ± 2946 | 100 | 90 ± 85 | 1805 ± 1693 | 100 |
| XXIIt-110 | 223 | 4466 | 87 | 155 ± 150 | 3097 ± 3005 | 100 |
| XXIII-120 | Not | Not | | 109 ± 39 | 2183 ± 785 | 100 |
| | significant | significant | | | | |
| XXIV-130 | Not | Not | | 260 ± 9 | 5199 ± 177 | 100 |
| | significant | significant | | | | |
| XXV-75 | Not | Not | | 118 | 2352 | 89 |
| | significant | significant | | | | |
| XXV-100 | Not | Not | | 37 ± 52 | 744 ± 1037 | 100 |
| | significant | significant | | | | |
| LI-120 | 189 ± 166 | 3783 ± 3311 | 37 | 56 ± 22 | 1124 ± 444 | 99 |
| HemPE80 | Not | Not | | | Not | |
| | significant | significant | | | significant | |
| XXVIII90 | Not | Not | | 347 | 6944 | 26 |
| | significant | significant | | | | |
| XLV | 5 | 101 | 60 | 25 ± 27 | 509 ± 536 | 56 |
| 40/100 | | | | | | |
| XLV | Not | Not | | | Not | |
| 10/60 | significant | significant | | | significant | |
| XLV 5/60 | Not | Not | | | Not | |
| | significant | significant | | | significant | |

### Example 9

### Toxicity of Lipid Conjugates

### Methods:

The lipid conjugates were prepared as described in Example 5. Lipid Toxicity to SMC cells was observed microscopically.

### Results:

The toxicity of representative compounds in human coronary artery smooth muscle cells (HCASMC) was assessed and results are presented in Table 8. Representative graph depicting the toxicity of lipid conjugates in smooth muscle cells is shown in FIG. 8.

TABLE 8

| compound | IC₅₀ (μg/mL) | IC₅₀ (nM) |
|---|---|---|
| XXIIf-110 | 2000 | 40,000 |
| XXIIt-110 | 2000 | 40,000 |
| XXIII-120 | 2000 | 40,000 |
| XXIV-130 | No toxicity observed | No toxicity observed |
| XXV-75 | No toxicity observed | No toxicity observed |
| XXV-100 | No toxicity observed | No toxicity observed |
| LI-120 | No toxicity observed | No toxicity observed |
| HemPE80 | No toxicity observed | No toxicity observed |
| XXVIII90 | No toxicity observed | No toxicity observed |
| XLV 40/100 | No toxicity observed | No toxicity observed |
| XLV 10/60 | No toxicity observed | No toxicity observed |
| XLV 5/60 | No toxicity observed | No toxicity observed |

### Example 10

### Embodiments of Synthesis Routes of Representative Lipid Conjugates

Preparation of Hy-Pe (Conjugation of Hyaluronic Acid with Phosphatidyl-ethanolamine)

Hyaluronic acid (HY) was truncated, and 15 g were dissoved in 9 L water, a solution of 150 mg FeSO₄.7H₂O in 20 mL water; 300 mL H₂O₂ (30%) were added to the reaction mixture and the reaction mixture was stirred for 1.5 h. The mixture was filtered through 30 Kd Filtron followed by lyophilization.

A solution of HY (1.2 g HY acid dissolved in 50 mL of 4-morpholineethanesulfonic acid (MES)-buffer (pH-6.5, 0.1M)) was mixed with a solution of PE (180 mg PE dissolved in 50 mL t-BuOH and 10 mL H₂O). N-Hydroxybenzotriazole (HOBt) (120 mg) and 1-ethyl-3-(3-dimethylaminopropyl) carbodiimide (EDC) (1.2 g) were added. The mixture was sonicated in an ultrasound bath for 3 h, dialyzed and lyophilized.

The content of the reactor was diluted with 3.5 liters of Process Water in reservoir via the recycle pump. 50 liters of Process Water containing 1.6% wt sodium bicarbonate (pH=8.0-8.3) was fed in at a rate of ca.6.5 liters/hour. The pressure during the two filtration stages was 20 PSI in the feed flow and about 7 PSI in the permeate flow. The filtrate flow was without pressure. The process was now repeated using Process Water only (67 liters) which was fed in at 10 liters/hour. The volume in the reservoir during the all filtrations was 5.0 liter. At the end of the last filtration the feed was closed and the volume in the reservoir was decreased to 1.2 liters, to give the final HyPE concentrate. The mixture in the reservoir must be stirred during the continuous dilution/filtration procedure to prevent formation of a concentration gradient which could cause inconsistent (and inefficient) washing.

Freeze drying (lyophilization) was carried out in a Sublimation Freeze Drying System Dura-Dry®, FTS® Systems, Inc. using Lyoguard® freeze drying trays. The HyPE concentrate (1.2 to 1.5 L) was added to the tray and cooled by the tray cooling system to a temperature of (−14) to (−15)° C. The temperature in the condenser was (−43) to (−42)° C. After freezing of the sample in the tray, a vacuum of 1-2 mbar (absolute pressure) was set by pump. The tray was heated by the control system to, initially, a temperature of +25° C. and then to +35° C. at the end of the ice sublimation. The duration of the freeze drying procedure was about 48-60 hours.

The above methods may be utilized for the preparation of any lipid conjugate compound of this invention, and modified as necessary, as will be appreciated by one skilled in the art.

Preparation of Hy-Dmpe (Conjugation of Hyaluronic Acid with Dimyristoyl phosphatidylethanolamine)

HY-DMPE was prepared according to the process of HY-PE, with thorough mixing. Sonication was optional.

Preparation of Ha-Dppe (Conjugation of Hyaluronic Acid with Dipalmitol-phosphatidylethanolamine)

20.0±0.1 g of hyaluronic acid (HA) 70-30 kDa (dry weight basis) was dissolved in 770±20 mL of the 0.1 M MES buffer (adjusted with 5N NaOH to pH=6.4) while stirring and heating to 35±5° C. (solution 1). 2.00±0.05 g of dipalmitoyl-phosphatidyl-ethanolamine (DPPE) was dissolved in a mixture of 750±20 mL tert-butanol (93% t-BuOH, 7% water) and 65±2 mL of water while stirring and heating to 50±5° C. until complete dissolution of the DPPE was achieved (solution 2).

Solution 2 was added to solution 1 under stirring. 2.00±0.05 g N-Hydroxybenzotriazole (HOBt) was added and the mixture was allowed to cool to 30±5° C. 20±0.1 g 1-ethyl-3-(3-dimethylaminopropyl) carbodiimide (EDC) was added and when dissolved (1-2 minutes) the reaction mixture was transferred to a 2 liter RB flask. The reaction mixture was sonicated in an ultrasound bath (TS-540) for 3 hours. The bath was cooled so that the temperature in the bath did hot exceed 35° C. Under these conditions, the temperature in the reaction mixture flask was kept between 35-39° C. On completion of the sonication step, the reaction mixture was stirred overnight at room temperature.

Extraction: Into the separation funnel was added the reaction mixture (approximately 1.6 L), 850 mL of dichloromethane (DCM) and 850 mL of methanol. The lower organic phase was mixed then separated. The extraction was repeated twice by adding 480 mL DCM and 240 mL of ethanol to the aqueous phase. After separation, the water phase was transferred to the 2 Liter glass reactor and residual organics were distilled out by heating up to a maximum temperature of 68° C. When the temperature in the reactor reached 65-66° C., a flow of nitrogen at approximately 200 L/min was passed through for 5-7 min. A test to check the DCM in the distillate was then carried out. If the test indicated the absence of DCM, the reactor was cooled to 30-35° C. and the content of the reactor was transferred to a reservoir of the membrane filtration system.

Preparation of Hem-Pe (Conjugation of Polygeline (Haemaccel) with Phosphatidylethanolamine)

Preparation of Hem-NH: 4 g hexanediamine was dissolved in 400 mL H₂O, titrated with HCl to pH=6.0. 500 mL of 3.5% haemaccel and 2 g of 1-Ethyl-3-(3-dimethyllaminopropyl)carbodiimide (EDC) were added. The solution was titrated with HCl to maintain the pH at 6.0. The reaction was titrated for 3-4 h and the reaction was left overnight. In the morning the pH was adjusted to 6.0, and 0.5 g EDC was added and the reaction continued for additional ˜4 hours. The reaction mixture was diluted to 3 L, and was acidified to pH=3.0-4.0 and filter through 10 Kd Filtron, as described above.

Binding Hem-NH to Glu-PE: 200 mg of glutaryl-phosphatidyl-ethanolamine (Glu-PE) were dissolved in chloroform/methanol:1/1. The solution was activated with 800 mg dicyclohexylcarbodiimide (DCC) for 1.5 hour. The solvents were evaporated in a rotor vacuum, and a solution of 1 g Hem-NH dissolved in 40 mL H₂O containing 1 mL didodecyldimethylammonium bromide (DiDAB) and 0.5 mL triethylamine was added immediately and was reacting for 48 h.

The reaction mixture was washed with dichloromethane, methanol and ethanol to remove free Glu-PE. The aqueous phase was dialyzed against water and was lyophilized. The reaction mixture was dissolve in a mixture of water and methanol 1:1, and was passed through an ion exchange column (Amberlite IR 120) followed by dialyzation against water and lyophilization.

Preparation of ChSA-Pe (Conjugation of Chondroitin Sulfate a with Phosphatidyl-ethanolamine)

A solution of Chondroitin Sulfate A (10 g Chondroitin Sulfate A acid dissolved in 1200 mL of 4-morpholineethanesulfonic acid (MES)-buffer (pH-6.5, 0.1M)) was mixed with a solution of PE (1.5 g PE dissolved in 120 mL of chloroform/methanol 1:1 with 15 mL 50% DiDAB in water/MeOH/EtOH*). The mixture was stirred thoroughly. 1 gr of HOBt was added, followed by addition of 10 g EDC (after 5-10 minutes). The mixture was stirred for 48 h.

Followed by the steps as described for Hem-PE.
*50% DiDAB in water/MeOH/EtOH is a commercially available solution Aldrich catalog. No. 33125-2.

It will be appreciated by persons skilled in the art that the present invention is not limited by what has been particularly shown and described herein above and that numerous modifications, all of which fall within the scope of the present invention, exist. Rather, the scope of the invention is defined by the claims which follow:

What we claim is:

1. A device having a coating on at least a portion of a surface of said device, said coating comprising a lipid or phospholipid moiety bound to a polypyranose.

2. The device according to claim 1, wherein said phospholipid moiety is phosphatidylethanolamine.

3. The device according to claim 2, wherein said phosphatidylethanolamine is dipalmitoyl phosphatidylethanolamine.

4. The device according to claim 2, wherein said phosphatidylethanolamine is dimyristoyl phosphatidylethanolamine.

5. The device according to claim 1, wherein said polypyranose is a glycosaminoglycan.

6. The device according to claim 5, wherein said glycosaminoglycan is hyaluronic acid.

7. The device according to claim 5, wherein said glycosaminoglycan is heparin.

8. The device according to claim 5, wherein said glycosaminoglycan is chondroitin sulfate.

9. The device according to claim 8, wherein said chondroitin sulfate is chondroitin-6-sulfate, chondroitin-4-sulfate or a derivative thereof.

10. The device according to claim 1, where said device is a stent.

11. The device according to claim 1, where said device is a catheter.

12. The device according to claim 1, wherein said polypyranose is carboxymethylcellulose.

13. The device according to claim 1, wherein said polypyranose is alginate.

14. The device according to claim 1, wherein said polypyranose is hydroxyethylstarch (HES).

15. The device according to claim 1, wherein said polypyranose is dextran.

16. The device according to claim 1, wherein said coating comprises a compound represented by the structure of the general formula (A): 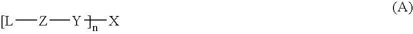
wherein
L is a lipid or a phospholipid;
Z is either nothing, ethanolamine, serine, inositol, choline, phosphate, or glycerol;
Y is either nothing or a spacer group ranging in length from 2 to 30 atoms;
X is a glycosaminoglycan; and
n is a number from 1 to 1000;
wherein any bond between L, Z, Y and X is either an amide or an esteric bond.

17. The device according to claim 16, wherein L is phosphatidylethanolamine.

18. The device according to claim 17, wherein said phosphatidylethanolamine is dipalmitoyl phosphatidylethanolamine.

19. The device according to claim 17, wherein said phosphatidylethanolamine is dimyristoyl phosphatidylethanolamine.

20. The device according to claim 16, wherein said glycosaminoglycan is hyaluronic acid.

21. The device according to claim 16, wherein said glycosaminoglycan is heparin.

22. The device according to claim 16, wherein said glycosaminoglycan is chondroitin sulfate.

23. The device according to claim 22, wherein said chondroitin sulfate is chondroitin-6-sulfate, chondroitin-4-sulfate or a derivative thereof.

24. The device according to claim 16, wherein said compound is represented by the structure of the general formula (I): 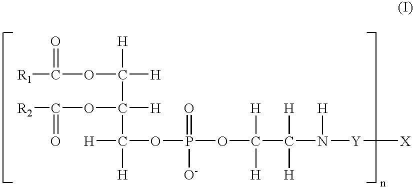
wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms; and
Y, X, and n are as defined hereinabove;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

25. The device according to claim 24, wherein R₁ and R₂ are palmitic acid moieties.

26. The device according to claim 24, wherein R₁ and R₂ are myristic acid moieties.

27. The device according to claim 24, wherein said glycosaminoglycan is hyaluronic acid.

28. The device according to claim 24, wherein said glycosaminoglycan is heparin.

29. The device according to claim 24, wherein said glycosaminoglycan is chondroitin sulfate.

30. The device according to claim 29, wherein said chondroitin sulfate is chondroitin-6-sulfate, chondroitin-4-sulfate or a derivative thereof.

31. The device according to claim 16, wherein said compound is represented by the structure of the general formula (II): 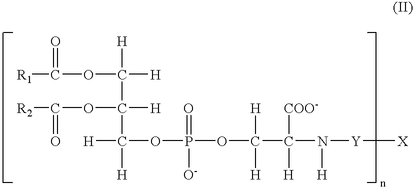
wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms; and
Y, X, and n are defined as in hereinabove;
wherein if Y is nothing the phosphatidylserine is directly linked to X via an amide bond and if Y is a spacer, said spacer is directly linked to X via an amide or an esteric bond and to said phosphatidylserine via an amide bond.

32. The device according to claim 16, wherein said compound is represented by the structure of the general formula (III): 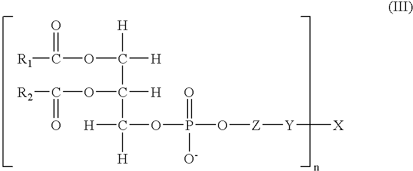
wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol; and
Y, X, and n are as defined hereinabove;
wherein any bond between the phosphatidyl, Z, Y and X is either an amide or an esteric bond.

33. The device according to claim 16, wherein said compound is represented by the structure of the general formula (IV): 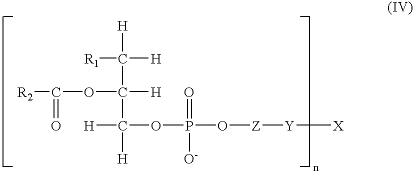
wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y, X, and n are as defined hereinabove;
wherein any bond between the phospholipid, Z, Y and X is either an amide or an esteric bond.

34. The device according to claim 16, wherein said compound is represented by the structure of the general formula (V): 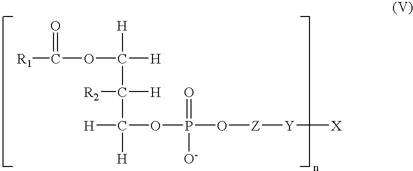
wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, inositol, choline, or glycerol;
Y, X, and n are as defined hereinabove;
wherein any bond between the phospholipid, Z, Y and X is either an amide or an esteric bond.

35. The device according to claim 16, wherein said compound is represented by the structure of the general formula (VI): 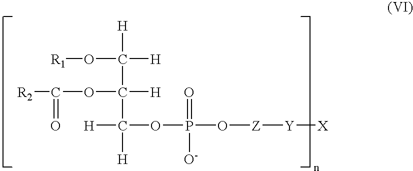
wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y, X, and n are as defined hereinabove;
wherein any bond between the phospholipid, Z, Y and X is either an amide or an esteric bond.

36. The device according to claim 16, wherein said compound is represented by the structure of the general formula (VII): 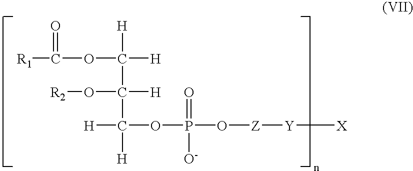
wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, inositol, choline, or glycerol;
Y, X, and n are as defined hereinabove;
wherein any bond between the phospholipid, Z, Y and X is either an amide or an esteric bond.

37. The device according to claim 16, wherein said compound is represented by the structure of the general formula (VIII): 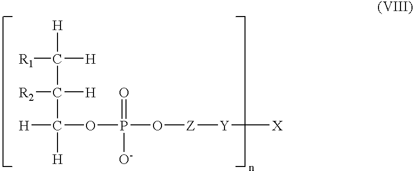
wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y, X, and n are as defined hereinabove;
wherein any bond between the phospholipid, Z, Y and X is either an amide or an esteric bond.

38. The device according to claim 16, wherein said compound is represented by the structure of the general formula (IX): 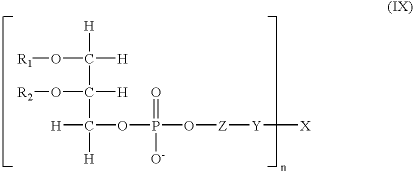
wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y, X, and n are as defined hereinabove;
wherein any bond between the phospholipid, Z, Y and X is either an amide or an esteric bond.

39. The device according to claim 16, wherein said compound is represented by the structure of the general formula (X): 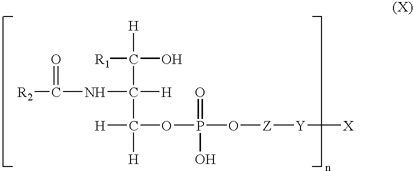
wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, ethaniolamine, serine, inositol, choline, or glycerol;
Y, X, and n are as defined hereinabove;
wherein any bond between the ceramide phosphoryl, Z, Y and X is either an amide or an esteric bond.

40. The device according to claim 16, wherein said compound is represented by the structure of the general formula (XI): 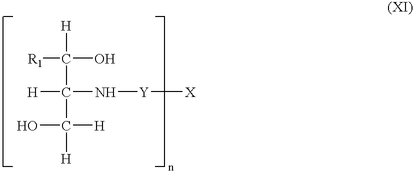
wherein
R₁ is a linear, saturated, mono-unsaturated, or poly-unsaturated, alkyl chain ranging in length from 2 to 30 carbon atoms;
Y, X, and n are as defined hereinabove;
wherein if Y is nothing the sphingosyl is directly linked to X via an amide bond and if Y is a spacer, said spacer is directly linked to X and to said sphingosyl via an amide bond and to X via an amide or an esteric bond.

41. The device according to claim 16, wherein said compound is represented by the structure of the general formula (XII): 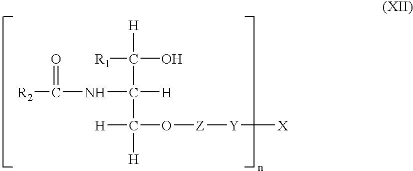
wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, ethanolamine, serine, inositol, choline, or glycerol;
Y, X, and n are as defined hereinabove;
wherein any bond between the ceramide, Z, Y and X is either an amide or an esteric bond.

42. The device according to claim 16, wherein said compound is represented by the structure of the general formula (XIII): 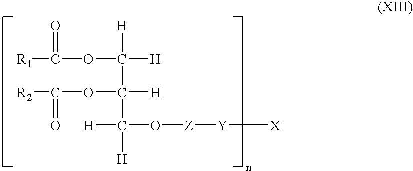
wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y, X, and n are as defined hereinabove;
wherein any bond between the diglyceryl, Z, Y and X is either an amide or an esteric bond.

43. The device according to claim 16, wherein said compound is represented by the structure of the general formula (XIV): 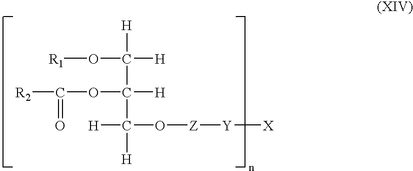
wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y, X, and n are as defined hereinabove;
wherein any bond between the glycerolipid, Z, Y and X is either an amide or an esteric bond.

44. The device according to claim 16, wherein said compound is represented by the structure of the general formula (XV): 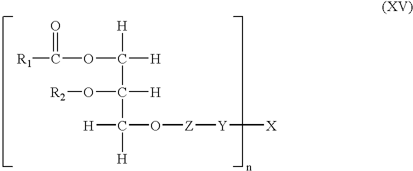
wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y, X, and n are as defined hereinabove;
wherein any bond between the glycerolipid, Z, Y and X is either an amide or an esteric bond.

45. The device according to claim 16, wherein said compound is represented by the structure of the general formula (XVI): 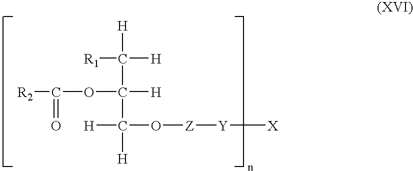
wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y, X, and n are as defined hereinabove;
wherein any bond between said lipid, Z, Y and X is either an amide or an esteric bond.

46. The device according to claim 16, wherein said compound is represented by the structure of the general formula (XVII): 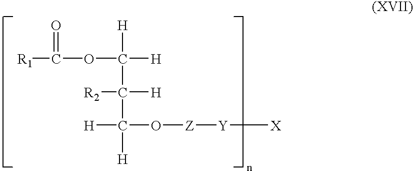
wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y, X, and n are as defined hereinabove;
wherein any bond between the lipid, Z, Y and X is either an amide or an esteric bond.

47. The device according to claim 16, wherein said compound is represented by the structure of the general formula (XVIII): 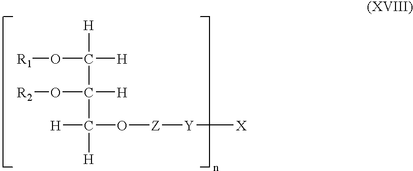
wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y, X, and n are as defined hereinabove;
wherein any bond between the lipid, Z, Y and X is either an amide or an esteric bond.

48. The device according to claim 16, wherein said compound is represented by the structure of the general formula (XIX): 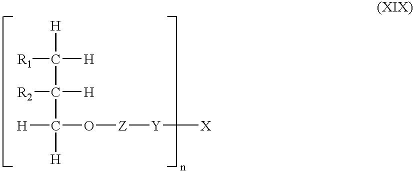
wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y, X, and n are as defined hereinabove;
wherein any bond between the lipid, Z, Y and X is either an amide or an esteric bond.

49. The device according to claim 16, wherein said compound is represented by the structure of the general formula (XX): 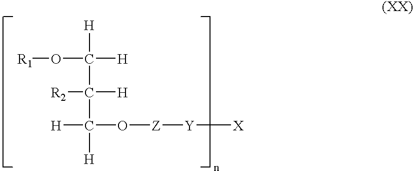
wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y, X, and n are as defined hereinabove;
wherein any bond between the lipid, Z, Y and X is either an amide or an esteric bond.

50. The device according to claim 16, wherein said compound is represented by the structure of the general formula (XXI): 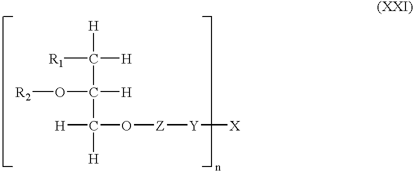
wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y, X, and n are as defined hereinabove;
wherein any bond between the lipid, Z, Y and X is either an amide or an esteric bond.

51. A method of preventing, inhibiting or treating vessel damage or vessel occlusion in a subject comprising the step of applying to said vessel a device having a coating on at least a portion of a surface of said device, said coating comprising a lipid or phospholipid moiety bound to a polypyranose.

52. The method according to claim 51, wherein said damage or occlusion is introduced or exacerbated by a medical procedure.

53. The method according to claim 52, wherein said medical procedure is catheterization, stent implantation, prosthesis attachment, artificial organ implantation, or a combination thereof.

54. The method according to claim 52, wherein said damage or occlusion is due to smooth muscle cell proliferation, a pathogenic infection, thrombosis, tissue ischemia, reperfusion injury, or a combination thereof.

55. The method according to claim 52, wherein said damage or occlusion is exacerbated by diabetes.

56. The method according to claim 52, wherein said phospholipid moiety is phosphatidylethanolamine.

57. The method according to claim 56, wherein said phosphatidylethanolamine is dipalmitoyl phosphatidylethanolamine.

58. The method according to claim 56, wherein said phosphatidylethanolamine is dimyristoyl phosphatidylethanolamine.

59. The method according to claim 51, wherein said polypyranose is carboxymethylcellulose.

60. The method according to claim 51, wherein said polypyranose is dextran.

61. The device according to claim 51, wherein said polypyranose is a glycosaminoglycan.

62. The device according to claim 61, wherein said glycosaminoglycan is hyaluronic acid.

63. The device according to claim 61, wherein said glycosaminoglycan is heparin.

64. The device according to claim 61, wherein said glycosaminoglycan is chondroitin sulfate.

65. The device according to claim 64, wherein said chondroitin sulfate is chondroitin-6-sulfate, chondroitin-4-sulfate or a derivative thereof.

66. The device according to claim 1, wherein said polypyranose is alginate.

67. The device according to claim 1, wherein said polypyranose is hydroxyethylstarch (HES).

68. The method according to claim 51, wherein said compound is represented by the structure of the general formula (A): 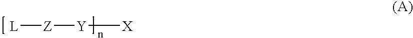
wherein
L is a lipid or a phospholipid;
Z is either nothing, ethanolamine, serine, inositol, choline, phosphate, or glycerol;
Y is either nothing or a spacer group ranging in length from 2 to 30 atoms;
X is a glycosaminoglycan; and
n is a number from 1 to 1000;
wherein any bond between L, Z, Y and X is either an amide or an esteric bond.

69. The method according to claim 68, wherein L is phosphatidylethanolamine.

70. The method according to claim 69, wherein said phosphatidylethanolamine is dipalmitoyl phosphatidylethanolamine.

71. The method according to claim 69, wherein said phosphatidylethanolamine is dimyristoyl phosphatidylethanolamine.

72. The method according to claim 68, wherein said glycosaminoglycan is hyaluronic acid.

73. The method according to claim 68, wherein said glycosaminoglycan is heparin.

74. The method according to claim 68, wherein said glycosaminoglycan is chondroitin sulfate.

75. The method according to claim 74, wherein said chondroitin sulfate is chondroitin-6-sulfate, chondroitin-4-sulfate or a derivative thereof.

76. The method of claim 52 wherein said vessel damage is due to stenosis or restenosis.
